(19) United States
(12) Patent Application Publication
(10) Pub. No.: US 2020/0172615 A1
(43) Pub. Date: Jun. 4, 2020

(54) NOVEL METHOD FOR PRODUCING ANTIBODIES

(71) Applicant: TSINGHUA UNIVERSITY, Beijing (CN)

(72) Inventors: Bai LU, Beijing (CN); Hongyang YAO, Beijing (CN); Wei GUO, Beijing (CN)

(21) Appl. No.: 16/611,856

(22) PCT Filed: May 8, 2018
(86) PCT No.: PCT/CN2018/085960

§ 371 Date: Nov. 8, 2019

(30) Foreign Application Priority Data
May 8, 2017 (CN) PCT/CN2017/083432
Jan. 12, 2018 (CN) PCT/CN2018/072469

(51) Int. Cl. C07K 16/28; C12N 5/078

(52) U.S. Cl. CPC C07K 16/28; C07K 2317/14; C12N 5/0634

(57) ABSTRACT

Disclosed is a method for producing an antibody or an antigen-binding fragment thereof comprising a step of cultivating PBMCs in a medium comprising CD40L, ICOSL, ICOS, and/or TLR agonist. Also provided herein is a method for inducing proliferation of PBMCs, B cell activation and differentiation, and/or B cell maturation, comprising a step of cultivating PBMCs in a medium comprising IL2. Also provided herein is a method for promoting class switch in an antibody-producing PBMC to produce IgG, comprising a step of cultivating the antibody-producing PBMC in a medium comprising IL21.

### FIELD OF THE INVENTION

The present disclosure generally relates to novel methods for producing antibodies, in particular in vitro method for producing fully human antibodies.

### BACKGROUND

Methods for producing antibodies are widely used in laboratory and clinics. Those include hybridoma technology, transgenic animal model and in vitro immunization. The traditional hybridoma technology is a mainstream mature technology, which includes steps of immunizing the animals, isolating lymphocyte, fusion of lymphocyte with immortalized cells such as myeloma, performing antibody humanization and affinity maturation. The antibodies can be produced in high throughput, but it has to face disadvantages including high cost, long production cycle, low affinity, unpredicted pair of heavy chain and light chain of the variable region. The transgenic animal model is a relatively new technology, where the animals are genetically modified to express human variable regions through unclear mechanisms. The in vitro immunization technology has been studied in recent years which does not require immunization of animals, and thus the process thereof are low in cost but faster and easier to operate, and the antibodies can be fully human without steps of humanization. However, few antibodies have been reported to be successfully generated using such methods. Therefore, there is a continuing need to develop new and effective methods for in vitro immunization to generate fully human antibodies.

### BRIEF SUMMARY OF THE INVENTION

In one aspect, the present disclosure provides a novel method for in vitro immunization to produce an antibody.

In certain embodiments, the method for producing an antibody or antigen-binding fragment thereof comprises a step of cultivating peripheral blood mononuclear cells (PBMCs) in a medium comprising at least one of the following: CD40 ligand (CD40L), Inducible T cell co-stimulator (ICOS), ICOS ligand (ICOSL), and/or Toll-like Receptor (TLR) agonists. In certain embodiments, the method for producing an antibody or antigen-binding fragment thereof comprises a step of cultivating PBMCs in a medium comprising both CD40L and ICOSL.

In certain embodiments, the medium further comprises IL2 and/or IL21. In certain embodiments, the PBMCs are isolated from a human, derived from hematopoietic stem cells (HSCs) or umbilical cord blood. In certain embodiments, the PBMCs comprises B cells and T follicular cells. In certain embodiments, the PBMCs comprises B cells and dendritic cells. In certain embodiments, the PBMCs comprises B cells, T follicular cells and dendritic cells.

In certain embodiments, the antibody or antigen-binding fragment thereof is human antibody or antigen-binding fragment thereof. In certain embodiments, the antibody is a monoclonal antibody.

In certain embodiments, the antibody or antigen-binding fragment thereof provided herein can be an affinity matured antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods.

The present disclosure provides that at least one of CD40L, ICOSL, ICOS, or TLR agonists can significantly increase the antibody production by PBMCs using the in vitro immunization provided herein, as compared with that of other cytokines or stimulants, such as CD40L alone. In certain embodiments, the TLR agonist is an agonist of TLR1, TLR2, TLR3, TLR4, TLR5, TLR6, TLR7, TLR8 or TLR9. In certain embodiments, the TLR agonist is a TLR7 and TLR8 (TLR7/8 or TLR7/TLR8) agonist. In certain embodiments, the TLR7 agonist is imiquimod. In certain embodiments, the TLR9 agonist is CpG ODN. In certain embodiments, the medium comprises ICOS and TLR agonist. In certain embodiments, the medium comprises CD40L and TLR agonist. In certain embodiments, the medium comprises ICOS and CD40L. In certain embodiments, the medium comprises ICOS, CD40L and TLR agonist.

In certain embodiments, the medium further comprises an antigen. The antigen is added to the medium at the beginning of the cultivation, or 1, 2, 3, 4, 5, 6, 7, 8, 9, 10, 11, 12, 13, 14, 15, 16, 17, 18, 19, 20, 21 or more days later. In certain embodiments, the antigen is present for at least 0.5 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25, one month or longer.

In certain embodiments, the medium further comprises stimulants including but not limited to co-stimulators, CpG ODN 2006 (CpG ODN), interleukins, anti-apoptotic proteins, tumor necrosis factors (TNFs), interferons (INFs), TLR Ligands, lipids, avasimid, EFNB 1, EPHB4, Plexin B2, Semaphorin 4C, B-lymphocyte-induced maturation protein (BLIMP-1), interferon regulatory factor 4 (IRF4), antibodies or any combination thereof. In certain embodiments, the co-stimulator is CD40, CD40L, ICOS, ICOSL, a proliferation-inducing ligand (APRIL), B cell activating factor of the TNF family (BAFF), OX40, OX40 Ligand (OX40L), or any combination thereof. In certain embodiments, the CpG ODNs are capable of stimulating toll-like receptor 9 (TLR9), including but not limited to CpG ODN 2006, D/K CpG, or any combination thereof. In certain embodiments, the interleukin includes, but not limited to IL2, IL21, IL4, IL5, IL6, IL7, IL10, IL13, IL14, IL15, IL33, or any combination thereof. In certain embodiments, the anti-apoptotic protein is Bcl-2, Bcl-6, Bcl-XL, Bcl-w, Mcl-1, analogs thereof or any combination thereof, which can be introduced into the PBMCs via known methods in the art, e.g. viral infection. In certain embodiments, the antibody can be anti-human IgG or anti-human IgM. In certain embodiments, the medium further comprises Ephrin-B 1 precursor (EFNB 1) and/or activation-induced cytidine deaminase (AICDA). In certain embodiments, the stimulants are derived from human or non-human animals. In certain embodiments, the stimulants are present in the medium at the start of the cultivation, or 1, 2, 3, 4, 5, 6, 7, 8, 9, 10, 11, 12, 13, 14, 15, 16, 17, 18, 19, 20, or 21 days later.

In certain embodiment, the stimulants are removed from the medium 1, 2, 3, 4, 5, 6, 7, 8, 9, 10, 11, 12, 13, 14, 15, 16, 17, 18, 19, 20, or 21 days later. In certain embodiment, the removal is by exchange of medium or washing the cultivated cells.

In certain embodiments, at least one of CD40L, ICOSL, ICOS and TLR agonists induces enhancement of antibody production by the PBMCs, B cell differentiation, and/or B cell maturation in the PBMCs.

In certain embodiments, the method further comprising a step of isolating an antibody secreted from the cultivated PBMCs. The isolation includes a step of fusion of the antibody-producing PBMCs with human myeloma cell line to generate hybridoma, or by isolating Fv clone variable domain gene sequences selected from human-derived display libraries (such as a phage display library, yeast display library or mammal cell display library). Such variable domain gene sequence may then be operably linked to a desired human constant domain gene sequence, and express, harvest and purify the antibody from the supernatant medium. In certain embodiments, the antibody-producing PBMC is B cell.

In certain embodiments, the method further comprising obtaining a nucleic acid sequence encoding a variable region of the antibody. In certain embodiments, the method further comprising introducing the nucleic acid sequence into a host cell under a condition suitable for expressing the antibody or antigen-binding fragment thereof. In certain embodiments, obtaining a nucleic acid sequence includes isolating the DNA or RNA fragment from a biological sample, such as a cell, a tissue or a blood sample, such as PBMCs. In certain embodiments, the nucleic acid sequence is a cDNA obtained via reverse transcription.

The present disclosure also provides a method for inducing proliferation of PBMCs, B cell differentiation, and/or B cell maturation, comprising a step of cultivating PBMCs in a medium comprising IL2. In certain embodiments, further comprising the presence of at least one of CD40L, ICOSL, ICOS and TLR agonists and/or IL21.

In certain embodiments, the medium does not contain IL2. In certain embodiments, more PBMCs are cultivated to have sufficient amount of B cells.

The present disclosure also provides a method for promoting class switch in an antibody-producing PBMC to produce IgG, comprising a step of cultivating the antibody-producing PBMC in a medium comprising IL21. In certain embodiments, the medium further comprises IL2 and/or at least one of CD40L, ICOSL, ICOS and TLR agonists. In certain embodiments, the antibody-producing PBMC is B cell.

In certain embodiments, the medium does not contain IL21. In certain embodiments, the class switch in an antibody-producing PBMCs to produce IgG occurs in the absence of IL21.

The present disclosure also provides a method for producing an antibody or antigen-binding fragment thereof comprising: cultivating PBMCs in the presence of IL2, at least one of CD40L, ICOSL, ICOS and TLR agonists, an antigen, IL21, and/or any combination thereof.

The present disclosure also provides a method for producing an antibody or antigen-binding fragment thereof comprising: a) cultivating PBMCs in a medium comprising IL2; b) adding at least one of CD40L, ICOSL, ICOS and TLR agonists, and an antigen to the medium; and c) adding IL21 to the medium. In certain embodiments, the medium further comprises stimulants including but not limited to co-stimulators, CpG oligodeoxynucleotides (CpG ODNs), interleukins, anti-apoptotic proteins, TNFs, interferons (INFs), TLR ligands, lipids, avasimid, EFNB1, EPHB4, Plexin B2, Semaphorin 4C, BLIMP-1, IRF4, antibodies or a combination thereof.

The present disclosure also provides a method for producing an antibody or antigen-binding fragment thereof comprising: a) cultivating PBMCs in a first medium comprising IL2; b) cultivating the PBMCs obtained in step a) in a second medium comprising at least one of CD40L, ICOSL, ICOS and TLR agonists and an antigen; and c) cultivating the PBMCs obtained in step b) in a third medium comprising IL21. In certain embodiments, the first, second and/or third medium further comprises stimulants including but not limited to co-stimulators, CpG ODNs, interleukins, anti-apoptotic proteins, TNFs, interferons (INFs), TLR ligands, lipids, avasimid, EFNB1, EPHB4, Plexin B2, Semaphorin 4C, BLIMP-1, IRF4, antibodies or a combination thereof.

In certain embodiments, the co-stimulator is CD40, CD40L, ICOS, ICOSL, APRIL, B cell activating factor of the TNF family (BAFF), OX40, OX40L, or any combination thereof. In certain embodiments, the CpG ODNs are capable of stimulating TLR9, including but not limited to CpG2006, D/K CpG, or a combination thereof. In certain embodiments, the interleukin includes, but not limited to IL2, IL21, IL4, IL5, IL6, IL7, IL10, IL13, IL14, IL15, IL33, or a combination thereof. In certain embodiments, the anti-apoptotic protein is Bcl-2, Bcl-6, Bcl-XL, Bcl-w, Mcl-1, analogs thereof or a combination thereof, which can be introduced into the PBMCs via known methods in the art, e.g. viral infection. In certain embodiments, the antibody can be anti-human IgG or anti-human IgM. In certain embodiments, the medium further comprises Ephrin-B 1 precursor (EFNB 1) and/or activation-induced cytidine deaminase (AICDA). In certain embodiments, the stimulants are derived from human or non-human animals.

In certain embodiments, the method further comprises obtaining a nucleic acid sequence encoding a variable region of the antibody; and optionally introducing the nucleic acid sequence into a host cell under a condition suitable for expressing the antibody or antigen-binding fragment thereof. In certain embodiments, the method further comprises isolating the antibody secreted by the host cell.

The present disclosure also provides an antibody produced according to the methods described herein. In certain embodiments, the antibody or antigen-binding fragment thereof binds specifically to TrkA. The complete cDNA sequence of TrkA has the GENBANK accession number of AB019488.2 and the amino acid sequence of human TrkA has the GENBANK accession number of BAA34355.1.

Also provided herein is a method for producing a chimeric antigen receptor (CAR), comprising a step of expressing a first nucleic acid operably linked to a second nucleic acid, wherein the first nucleic acid encodes an antigen binding domain derived from the antibody or antigen-binding fragment thereof produced according to the method or the antibody described herein, and wherein the second nucleic acid encodes a T-cell signaling domain.

Also provided herein is a method of treating a cancer in a subject comprising expressing in a T cell a first nucleic acid operably linked to a second nucleic acid, wherein the first nucleic acid encodes an antigen binding domain derived from the antibody or antigen-binding fragment thereof produced according the method or the antibody described herein, and wherein the second nucleic acid encodes a T-cell signaling domain; and administering the T cell to the subject. In certain embodiments, the T cell is optionally obtained from the subject.

In certain embodiments, a first batch of one or more of the stimulants are added to the medium for a first period of time after the beginning of the cultivation, followed by addition to the medium a second batch of one or more of the stimulants for a second period of time. In certain embodiments, said first batch of one or more of the stimulants are removed before addition of the second batch of one or more of the stimulants. In certain embodiments, said second batch of one or more of the stimulants are removed at the end of the second period. In certain embodiments, said “first period” or “second period” refers to, e.g. 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer. In certain embodiments, the “first period” or “second period” are of the same or different length (or time span). In certain embodiments, the first batch of one or more of the stimulants and the second batch of one or more of the stimulants are added at the same time. In certain embodiments, the first batch and second batch of one or more of the stimulants are of the same or different stimulants. In certain embodiments, the first batch of one or more of the stimulants is IL2, and the second batch of one or more of the stimulants is ICOS, ICOSL, CD40L together with ICOSL, and/or TLR agonist.

In certain embodiments, a first batch of one or more of the stimulants are added to the medium for a first period of time after the beginning of the cultivation, followed by addition to the medium a second batch of one or more of the stimulants for a second period of time, then followed by addition to the medium a third batch of one or more of the stimulants for a third period of time. In certain embodiments, said first batch of one or more of the stimulants are removed before addition of the second batch of one or more of the stimulants. In certain embodiments, said second batch of one or more of the stimulants are removed before addition of the third batch of one or more of the stimulants. In certain embodiments, at least two batches of one or more of the stimulants are present in the medium. In certain embodiments, said “first period”, “second period” or “third period” refers to, e.g. 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer. In certain embodiments, the “first period”, “second period” or “third period” are of the same or different length (or time span). In certain embodiments, the first batch, second batch and third batch of one or more of the stimulants are of the same or different stimulants. In certain embodiments, the first batch of one or more of the stimulants is IL2, the second batch of one or more of the stimulants is ICOS, ICOSL, CD40L together with ICOSL, and/or TLR agonist, and the third batch of one or more of the stimulants is IL21.

In certain embodiments, the stimulants are present at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more g/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM. In certain embodiments, the IL2 is present at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more g/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM. In certain embodiments, the CD40L, ICOSL, ICOS, and/or TLR agonist is present at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500, 600, 700, 800, or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more μg/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM. In certain embodiments, the IL21 is present at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500, 600, 700, 800, 1000 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more g/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM. In certain embodiments, the CpG ODN is present at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500, 600, 700, 800, 1000 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more μg/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM.

In certain embodiments, the concentration of IL2 is 10 ng/ml. In certain embodiments, the concentration of IL21 is 50 ng/ml. In certain embodiments, the concentration of ICOS is 2 μg/ml. In certain embodiments, the concentration of ICOSL is 50 ng/ml, and/or CD40L is 2 μg/ml. In certain embodiments, the concentration of TLR agonist is 2 μg/ml, or 0.1 nM, 50 nM or 500 nM. The TLR agonist is TLR9 agonist CpG ODN at 2 μg/ml, or TLR7 agonist imiquimod or a synthesized TLR7/8 agonist at 50 nM or 500 nM.

In certain embodiments, the IL2 and IL21 are present in the concentration of a ratio of 1:1, 1:2, 1:5, 1:10, 1:20, 1:30, 1:40, 1:50, 1:60, 1:70, 1:80, 1:90, 1:100, 1:150, 1:200, 1:500, 1:1000, 1:2000, 1:5000, 1:10000, or 1:20000. In certain embodiments, the IL2, IL21 and ICOS are present in the concentration of a ratio of 1:5:10, 1:5:20, 1:5:30, 1:5:40, 1:5:50, 1:5:100, 1:5:200, 1:5:500, 1:5:1000, 1:5:1500, 1:5:2000, 1:5:5000, 1:5:10000, 1:5:20000, 1:5:50000, respectively. In certain embodiments, the IL2, IL21 and TLR agonist are present in the concentration of a ratio of 1:5:50, 1:5:100, 1:5:200, 1:5:500, 1:5:1000, 1:5:1500, 1:5:2000, 1:5:5000, 1:5:10000, 1:5:20000, 1:5:50000, respectively. In certain embodiments, the ICOSL and CD40L are present in the concentration of a ratio of or 1:1, 1:2, 1:5, 1:10, 1:20, 1:30, 1:40, 1:50, 1:60, 1:70, 1:80, 1:90, 1:100, 1:150, 1:200, 1:500, 1:1000, 1:2000, 1:5000, 1:10000, or 1:20000. In certain embodiments, the IL2, IL21 and CpG ODN 2006 are present in the concentration of a ratio of 1:5:10, 1:5:20, 1:5:30, 1:5:40, 1:5:50, 1:5:100, 1:5:200, 1:5:500, 1:5:1000, 1:5:1500, 1:5:2000, 1:5:5000, 1:5:10000, 1:5:20000, 1:5:50000, respectively.

In certain embodiments, the stimulants are present for at least 0.5 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer.

In certain embodiments, the IL2 is present for at least 0.5 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer.

In certain embodiments, the ICOSL, CD40L, ICOS, and/or TLR agonist is present for at least at least 0.5 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer.

In certain embodiments, the IL21 is present for at least 0.5 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer.

### BRIEF DESCRIPTION OF FIGURES

FIG. 1 illustrates that IL2 stimulates PBMC proliferation. PBMCs were immunized in vitro with the antigen TrkA (2 μg/ml) in the presence of various stimulants as indicated for 14 days. Cell density was counted by Hemocytometer. Note that treatment with IL2 increased cell density by 15 folds. Concentration of the stimulants added: avasmibe, 10 μM/ml; CpG ODN, 2 μg/ml; IL21, 50 ng/ml; IL2, 10 ng/ml; IL4, 10 ng/ml; BAFF, 50 ng/ml. The concentrations of ICOSL are indicated in the figure.

FIG. 2A-2B illustrate that ICOSL together with CD40L strongly stimulates antibody IgG but not IgM production from B cells within the PBMCs after in vitro immunization. PBMCs were cultured in medium with various stimulants for 7 days as indicated. The TrkA protein was added into the medium as the antigen, together with the stimulants, on day 0. The production of anti-TrkA antibody at day 7 in the form of IgG (FIG. 2A) or IgM (FIG. 2B) was examined with an ELISA assay. Note that in the presence of IL2 (10 ng/ml) and IL21 (50 ng/ml) (also known as basic) plus CpG ODN, CD40L and ICOSL together elicited a much stronger stimulation to the production of anti-TrkA antibody IgG but not IgM, compared with either CD40L or ICOSL alone. Concentration of the stimulants added: CD40L, 2 μg/ml; OX40L, as indicated in the figure; ICOSL, 2 μg/ml. All other stimulants, the same as those in FIG. 1.

FIG. 3A-3B show that among all individual stimulants tested, only IL21 stimulated the production of antibody IgG but not IgM. PBMCs were immunized with the antigen TrkA, together with the individual stimulants as indicated, for 7 days. The production of anti-TrkA antibody at day 7 in the form of IgG (FIG. 3A) or IgM (FIG. 3B) was examined with ELISA assay. Concentrations of the stimulants added were the same as FIG. 2. Cholesterol, 5 μg/ml.

FIGS. 4A-4B show that CD40L or ICOS enhanced the antigen-induced production of antibody IgG (4A) or IgM (4B) by in vitro immunization. PBMCs were immunized with the antigen ovalbumin (OVA) or TrkA, and cultured in various conditions as indicated. The production of antibodies (anti-OVA or anti-TrkA) in the form of IgG (FIG. 4A) or IgM (FIG. 4B) was measured by ELISA assays. Vehicle was PBS. Note that for either OVA or TrkA as an antigen. ICOS is more effective than CD40L in stimulating the production of the antibody IgG.

FIGS. 5A-5C are the FACS results showing the germinal center (GC) like features (CD3−, CD19+, GL7+, Fas+) of B cells after in vitro immunization. FIG. 5A shows that there were very few GC like B cells in the absence of antigen or stimulants. FIG. 5B and FIG. 5C show that CD40L and ICOS, respectively, dramatically increased the generation of GC like B cells. In both cases, PBMCs were immunized with the antigen OVA (2 μg/ml), cultured in the presence of IL2+IL21 (basic). CD40L (55 nM) or ICOS (55 nM) was added to the culture media at the same time as basic. The cells were sorted and counted by the FACS machine.

FIGS. 6A and 6B show that toll like receptor (TLR) agonists are far superior to CD40L in stimulating the production of antibodies. PBMCs were immunized with the antigen OVA in the presence of “basic” (IL2 and IL21), and either CD40L (55 nM) or synthesized TLR7/8 agonist (50 or 500 nM) was added to the culture media together with the basic. The production of anti-OVA antibody, either in the form of IgG (FIG. 6A) or IgM (FIG. 6B) was measured by ELISA. Vehicle was PBS.

FIGS. 7A and 7B show antibody production by in vitro immunization in different donors. PBMCs were challenged by the antigen OVA in IL2 and L21 for 14 days, with either CD40L or a synthesized TLR7/8 agonist. FIG. 7A shows the IgG production and FIG. 7B shows the IgM production.

FIG. 8 shows the enhancement of AICDA (activation-induced cytidine deaminase, a gene known to be involved in antibody affinity maturation) expression by synthesized TLR7/8 agonist. The PBMCs derived from donor 3 and donor 4, respectively, were immunized by the antigen OVA (2 μg/ml) in basic with either CD40L (55 nM) or a synthesized TLR7/8 agonist (500 nM) for 14 days. Cells were harvested and the levels of AICDA were examined by RT-PCR. Vehicle is PBS.

FIG. 9 shows the effects of various stimulants on the expression of AICDA and BLIMP-1. PBMCs were immunized with the antigen OVA, and cultured in the presence of the stimulants indicated, and AICDA and BLIMP-1 were measured by RT-PCR the same way as above.

FIGS. 10A and 10B show that a TLR9 agonist has similar effect as CD40L in stimulating anti-OVA antibody production in PBMCs. Experiments were carried out the same way as FIG. 6, and the antibody production was measured on day 14 by ELISA assay. FIG. 10A shows the IgG production, and FIG. 10B shows the IgM production.

FIGS. 11A-11G show the interactive effects between synthesized TLR7/8 agonist and TLR9 antagonist in stimulating anti-OVA antibody production in PBMCs. At low concentrations (0.02-0.2 uM), the TLR9 antagonist E6446 enhanced the effect of synthesized TLR7/8, whereas at a high concentration (10 uM), E6446 inhibited this effect. Enzyme-linked immunosorbent assay (ELISA) analysis of OVA-specific antibodies of IgG (FIGS. 11A and 11D) and IgM (FIGS. 11B and 11E) responses were performed 7 days or 14 days after stimulant incubation. Cell proliferation (FIGS. 11C and 11F) was assayed by CellTiter-glo kit. Cells were harvested and tested by Flow cytometry (G). Follicular dendritic cells (FDCs) cells were gated as CD3⁻CD19⁻CD21⁺/CD35⁺ cells. Note: CD35 and CD21 are dendritic cell (DC) markers which may represent two subpopulations of DCs. The CD21-sub-type is inhibited by high concentration of E6446. Basic was referred to as OVA+IL2+IL21. The data represented the mean of 3 replicates; error bars represented SD. One representative data of 3 separate experiments was shown. *, p<0.05 for stimulation with basic+TLR7/8 agonist-500 nm+E6446-0.2 uM vs stimulation with only basic+TLR7/8 agonist-500 nm in IgG responses. **, p<0.05 for stimulation with basic+TLR7/8 agonist-500 nm vs stimulation with basic in IgG responses. *** p<0.001 for stimulation with basic+TLR7/8 agonist-500 nm+E6446-10 uM vs stimulation with basic+TLR7/8 agonist-500 nm in both IgG and IgM responses.

FIGS. 12A-12I show the synergistic and complementary effects of different stimulants on IgG and IgM responses. ELISA analysis of OVA-specific antibodies of IgG (FIGS. 12B, 12E, and 12H) and IgM (FIGS. 12A, 12D, and 12G) responses were performed 7 days or 14 days after stimulant incubation. Cell proliferation (FIGS. 12C, 12F, and 12I) was assayed by CellTiter-glo kit. Basic was referred as OVA+IL2+IL21. FIGS. 12A-12C: ICOS enhanced the effects of IL2 or IL21 on IgG production. FIGS. 12D-12F: CD40L enhanced the effects of IL2 or IL21 on IgG production. FIGS. 12G-12I: TLR7/8 enhanced the effects of IL2 or IL21 on IgG production. The data represented the mean of 3 replicates; error bars represented SD. One representative data of 3 separate experiments was shown. *, p<0.05 for stimulation with basic vs stimulation with basic+24 nM ICOS in IgG responses. **, p<0.05 for stimulation with basic vs stimulation with basic+55 nM CD40L or 500 nM synthesized TLR7/8 agonist in IgG responses. ****, p<0.0001 for stimulation with basic vs stimulation with basic+24 nM ICOS or 500 nM synthesized TLR7/8 agonist at day14 in IgG responses.

FIGS. 13A-13F show that ICOS, CD40L, synthesized TLR7/8 agonist regulated IgG and IgM responses in a dose-dependent manner. ELISA analysis of OVA-specific antibodies of IgG (FIGS. 13B, 13D, and 13F) and IgM (FIGS. 13A, 13C, and 13E) responses were performed 7 days or 14 days after stimulant incubation. Cell proliferation (FIGS. 13C, 13F, and 13I) was assayed by CellTiter-glo kit. Basic was referred to as OVA+IL2+IL21. The data represented the mean of 3 replicates; error bars represented SD. One representative data of 3 separate experiments was shown. **, p<0.05 for stimulation with basic vs stimulation with basic+100 nM ICOS or 500 nM synthesized TLR7/8 agonist in IgG responses or basic+24 nM CD40L in IgM responses, respectively. ***, p<0.001 for stimulation with basic vs stimulation with basic+24 nM CD40L in IgM responses. ****, p<0.0001 for stimulation with basic vs. stimulation with basic+100 nM ICOS or synthesized TLR7/8 agonist (50 nM and 500 nM) in IgM responses.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, multispecific antibody, or bispecific (bivalent) antibody or a functional portion thereof that binds to a specific antigen. A native intact antibody comprises two heavy chains (H) and two light (L) chains inter-connected by disulfide bonds. Each heavy chain consists of a variable region (VH) and a first, second, and third constant region (CH1, CH2 and CH3, respectively), while each light chain consists of a variable region (VL) and a constant region (CL). Mammalian heavy chains are classified as α, δ, ε, γ, and μ, and mammalian light chains are classified as λ or κ. The variable regions of the light and heavy chains are responsible for antigen binding. The variables region in both chains are generally subdivided into three regions of hypervariability called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Therefore, each VH and VL comprises of three CDRs and four FRs in the following order (amino acid residues N terminus to C terminus): FR1, CDR1, FR2, CDR2, FR3, CDR3, FR4. The constant regions of the heavy and light chains are not involved in antigen binding, but exhibit various effector functions. Antibodies are assigned to the five major classes based on the amino acid sequence of the constant region of their heavy chain: IgA, IgD, IgE, IgG, and IgM, which are characterized by the presence of α, δ, ε, γ, and μ heavy chains, respectively. Subclasses of several of the major antibody classes are such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized antibody” used herein refers to an antibody or antigen-binding fragment comprises CDRs derived from non-human animals, FR regions derived from human, and when applicable, constant regions derived from human.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “bivalent” as used herein refers to an antibody or an antigen-binding fragment having two antigen-binding sites; the term “monovalent” refers to an antibody or an antigen-binding fragment having only one single antigen-binding site; and the term “multivalent” refers to an antibody or an antigen-binding fragment having multiple antigen-binding sites. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse or rat. In some embodiments, the non-human animal is a mammal, for example, a mouse, a rat, a rabbit, a goat, a sheep, a guinea pig, or a hamster.

An “affinity matured” antibody refers to an antibody with one or more alterations or substitutions with amino acid residues in one or more hypervariable regions (HVRs), such as the complementarity determining regions (CDRs), compared to a parent antibody without such alterations or substitutions, which confer an improvement in the affinity of the antibody for antigen.

The term “substitution” with regard to amino acid residue as used herein refers to naturally occurring or induced replacement of one or more amino acids with another in a peptide, polypeptide or protein. Substitution in a polypeptide may result in diminishment, enhancement, or elimination of the polypeptide's function.

Substitution can also be “conservative substitution” with reference to amino acid sequence refers to replacing an amino acid residue with a different amino acid residue having a side chain with similar physiochemical properties or substitution of those amino acids that are not critical to the activity of the polypeptide. For example, conservative substitutions can be made among amino acid residues with nonpolar side chains (e.g. Met, Ala, Val, Leu, and Ile, Pro, Phe, Trp), among residues with uncharged polar side chains (e.g. Cys, Ser, Thr, Asn, Gly and Gln), among residues with acidic side chains (e.g. Asp, Glu), among amino acids with basic side chains (e.g. His, Lys, and Arg), among amino acids with beta-branched side chains (e.g., Thr, Val and Ile), among amino acids with sulfur-containing side chains (e.g., Cys and Met), or among residues with aromatic side chains (e.g. Trp, Tyr, His and Phe). In certain embodiments, substitutions, deletions or additions can also be considered as “conservative substitution”. The number of amino acids that are inserted or deleted can be in the range of about 1 to 5. Conservative substitution usually does not cause significant change in the protein conformational structure, and therefore could retain the biological activity of a protein.

As used herein, the term “antigen-binding fragment” refers to an antibody fragment formed from a fragment of an antibody comprising one or more CDRs, or any other antibody portion that binds to an antigen but does not comprise an intact native antibody structure. In certain embodiments, the antibody provided herein is an antigen-binding fragment. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, an isolated CDR and a bivalent domain antibody. An antigen-binding fragment is capable of binding to the same antigen to which the parent antibody binds. In certain embodiments, an antigen-binding fragment may comprise one or more CDRs from a particular human antibody.

An “antigen” or “Ag” as used herein refers to a compound, composition, peptide, polypeptide, protein, RNA, DNA, or substance that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, and CDC, but does not function in antigen binding.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc NatlAcad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'S of one moiety coordinate with the V_{L}'S of the other moiety and form two binding sites which can target the same antigens (or epitopes) or different antigens (or epitopes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a VL domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. A bivalent molecule can be monospecific if the two binding sites are both for specific binding of the same antigen or the same epitope. Similarly, a trivalent molecule can be bispecific, for example, when two binding sites are monospecific for a first antigen (or epitope) and the third binding site is specific for a second antigen (or epitope).

An “epitope” or “antigenic determinant” refers to the region of an antigen to which a binding agent (such as an antibody) binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “fully human” as used herein, with reference to antibody or antigen-binding fragment, means that the antibody or the antigen-binding fragment has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

“Substantially”, “substantially the same” as used herein refer to a high degree of similarity between two numeric values, and those skilled in the art would not recognize or consider a significant difference between the two values or of little difference with regard to statistics and/or biological activity as indicated by the values. In contrast, “substantially lower” means that a numeric value is less than about 50%, less than about 40%, less than about 30%, less than about 20%, less than about 10% as a function of the reference value.

The term “specific binding” or “specifically binds” as used herein refers to a non-random binding reaction between two molecules, such as for example between an antibody and an antigen. In certain embodiments, the antibodies or antigen-binding fragments provided herein specifically bind human and/or non-human antigen with a binding affinity (K_{D}) of about 0.01 nM to about 100 nM, about 0.1 nM to about 100 nM, 0.01 nM to about 10 nM, about 0.1 nM to about 10 nM, 0.01 nM to about 5 nM, about 0.1 nM to about 5 nM, 0.01 nM to about 1 nM, about 0.1 nM to about 1 nM or about 0.01 nM to about 0.1 nM). K_{D} as used herein refers to the ratio of the dissociation rate to the association rate (k_{off}/kₒₙ), may be determined using surface plasmon resonance methods for example using instrument such as Biacore.

“Cancer” or “cancerous condition” as used herein refers to any medical condition mediated by neoplastic or malignant cell growth, proliferation, or metastasis, and includes both solid cancers and non-solid cancers such as leukemia. “Tumor” as used herein refers to a solid mass of neoplastic and/or malignant cells.

“Treating”, “treatment” or “therapy” of a condition as used herein can be used interchangeably, and includes therapeutic treatment, prophylactic or preventative measures, such as preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. With regard to cancer, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” includes eradicating all or part of a tumor, inhibiting or slowing tumor growth and metastasis, preventing or delaying the development of a tumor, or some combination thereof.

An “isolated” substance has been altered by the hand of man from the natural state. If an “isolated” composition or substance occurs in nature, it has been changed or removed from its original environment, or both. For example, an “isolated” polynucleotide or polypeptide is a polynucleotide or a polypeptide that is free of other polynucleotides or polypeptides, respectively, and is not associated with naturally components that accompany the polynucleotide or a polypeptide in the native state. In certain embodiments, an “isolated” antibody is purified by at least one step to a purity of at least 90%, 91%, 92%, 93%, 94%, 95%, 96%, 97%, 98%, 99% as determined by electrophoretic methods (such as SDS-PAGE using Coomassie blue or silver stain, isoelectric focusing, capillary electrophoresis), chromatographic methods (such as ion exchange chromatography or reverse phase HPLC) or Lowry method.

The term “vector” as used herein refers to a vehicle into which a polynucleotide encoding a protein may be operably inserted and transported so as to express that protein in a host cell. A vector may be used to transform, transduce, or transfect a host cell so as to bring about the expression of the genetic element it carries within the host cell. Exemplary types of vectors includes, but not limited to, plasmids (e.g. phagemids, cosmids, yeast artificial chromosome (YAC), bacterial artificial chromosome (BAC) or P1-derived artificial chromosome (PAC)), viral vector (bacteriophages such as lambda phage or M13 phage, or animal viruses), bacterial vector, or non-episomal mammalian vectors. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector (e.g. a bacterial vector or episomal mammalian vector) may contain an origin of replication. A vector may also include materials to aid in its entry into the cell, including but not limited to a viral particle, a liposome, or a protein coating.

A “nucleic acid” or a “nucleic acid sequence” or “polynucleotide”, can be used interchangeably herein, refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The “host cell” as used herein refers to a cell into which an exogenous polynucleotide and/or a vector has been introduced to express one or more exogenous proteins. It intends to refer to both the particular subject cell and the progeny thereof. A host cell can be a prokaryote, a eukaryote, a plant cell, an animal cell or a hybridoma. It can be a cell that does not express a protein at a desired level but comprises the nucleic acid, unless a regulatory agent is introduced into the cell or a regulatory sequence is introduced into the host cell so that it is operably linked with the nucleic acid.

The term “mononuclear cells (MCs)” refers to neonatal cord blood mononuclear cell (CBMCs) and/or adult peripheral blood mononuclear cell (PBMCs). The term “total population of human peripheral blood mononuclear cells, total PBMC population, PBMCs, total PBMCs, or human PBMCs” are any peripheral blood cell having a round nucleus, comprising lymphocytes (T cells, B cells, NK cells, dendritic cells) and monocytes. The PBMC can be extracted from whole blood by conventional techniques in the art, such as density gradient centrifugation using ficoll, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes. Proliferation of PBMCs can be detected or confirmed in vitro by methods known in the art, for example, by MTT assay (a colorimertic method), AO/PI (Acridine Orange and Propidium Iodide) staining, or cell counting.

In certain embodiments, the PBMCs comprise B cells. In certain embodiments, the PBMCs comprise at least one type of B cells, T cells (e.g. T follicular cell), dendritic cells, NK cells, monocytes and any combination thereof. For example, in certain embodiments, the PBMCs comprise B cells and T cells (e.g. T follicular cell). In certain embodiments, the PBMCs comprise B cells and dendritic cells. In certain embodiments, the PBMCs comprise B cells, T cells (e.g. T follicular cell), and dendritic cells. In certain embodiments, the PBMCs comprise B cells and NK cells. In certain embodiments, the PBMCs comprise B cells and monocytes. In certain embodiments, the PBMCs comprise B cells, T cells (e.g. T follicular cell), and NK cells. In certain embodiments, the PBMCs comprise B cells, T cells (e.g. T follicular cell), dendritic cells and NK cells.

The term “B cell” as used herein refers to B lymphocytes, a type of white blood cell of the lymphocyte subtype. They function in the humoral immunity component of the adaptive immune system by secreting antibodies. B cells also present antigen and secrete cytokines. In mammals, B cells mature in the bone marrow. After B cells mature in the bone marrow, they migrate through the blood to secondary lymphoid organs (SLOs), such as the spleen and lymph nodes, where B cells receive a constant supply of antigen through circulating lymph. Unlike the other two classes of lymphocytes, i.e. T cells and natural killer cells, B cells express B cell receptors (BCRs) on their cell membrane, which allow the B cell to bind a specific antigen, against which it will initiate an antibody response. Of the three B cell subsets, FO B cells preferentially undergo T cell-dependent (TD) activation while marginal zone (MZ) B cells and B1 B cells preferentially undergo T cell-independent (TI) activation. B cells activated by TI antigens proliferate outside of lymphoid follicles but still in SLOs, possibly undergo immunoglobulin class switching, and differentiate into short-lived plasmablasts that produce early, weak antibodies mostly of class IgM, but also some populations of long-lived non-proliferating antibody-producing plasma cells. B cell activation is enhanced through the activity of CD21, a surface receptor in complex with surface proteins CD19 and CD81 (all three are collectively known as the B cell co-receptor complex, or BCR). When a BCR binds an antigen tagged with a fragment of the C3 complement protein, CD21 binds the C3 fragment, co-ligates with the bound BCR, and signals are transduced through CD19 and CD81 to lower the activation threshold of the cell. In certain embodiments, the B cells are those naturally exist in the PBMCs from a healthy donor.

The term “naive B lymphocytes” is intended to mean B lymphocytes (B cells) which have never encountered the antigen that they could bind via the paratope expressed by their surface immunoglobulin. These B cells are derived directly from the peripheral blood of a subject who has never been in contact with the antigen. These subjects will therefore exhibit a seronegative status with respect to said antigen, i.e. they will exhibit an undetectable titer of serum antibodies specific for said antigen.

“B cell development” as used herein refers to differentiation of lymphoid precursor cells differentiate into the earliest distinctive B-lineage cell (the progenitor B cell (pro-B cell)), which expresses a transmembrane tyrosine phosphatase, CD45R (or B220 in mice). Proliferation and differentiation of pro-B cells into precursor B cells (pre-B cells) requires the microenvironment provided by the bone marrow stromal cells, which interact directly with pro-B and pre-B cells, and secrete various cytokines, notably IL-7, that support the developmental process.

“B cell maturation” as used herein refers to a period which depends on rearrangement of the immunoglobulin DNA in the lymphoid stem cells. During B-cell development, sequential Ig-give rearrangements transform a pro-B cell into an immature B cell expressing mIgM with a single antigenic specificity. Future development yields mature naïve B cells, still of a single specificity, expressing both mIgM and mIgD. Only pre-B cells that are able to express membrane-bound μ heavy chains in association with surrogate light chains are able to proceed along the maturation pathway. Following the establishment of an effective pre-B cell receptor, each pre-B cell undergoes multiple cell divisions, perhaps six to eight, producing as many as 256 descendants. Each of these progeny pre-B cells may then rearrange different light-chain gene segments, thereby increasing the overall diversity of the antibody repertoire. In certain embodiments, the B cell maturation occurs in periphery. B cell maturation can be detected or confirmed in vitro by methods known in the art, for example, by detecting B cell surface markers, for example, immature B cells express mIgM and mIgD, and mature B cells express mIgG, mIgA and mIgD. Those skilled in the art will appreciate that methods such as cell staining and cell sorting with labeled antibodies against the above markers can be used. “B cell activation and differentiation” as used herein refers to a process of B lymphocyte in periphery undergoes antigen-induced activation and differentiation. Activated B cells can give-rise to antibody-secreting plasma cells or memory B cells. The class switch occurs at the stage of plasma cells. B cells may first differentiate into a plasmablast-like cell, then differentiate into a plasma cell, which are generated later in an infection and, compared to plasmablasts, have antibodies with a higher affinity towards their target antigen due to affinity maturation in the germinal center (GC) and produce more antibodies (see Nutt et al., Nature Reviews Immunology. 2015, 15 (3): 160). Plasma cells typically result from the germinal center reaction from T cell-dependent (TD) activation of B cells, however they can also result from T cell-independent (TI) activation of B cells (see Bortnick et al., The Journal of Immunology. 188 (11): 5389-5396). B cell activation or differentiation can be detected or confirmed in vitro by methods known in the art, for example, by cell labelling with CD19, IgM, IgD, IgA antibodies and cell sorting using FACS. Memory B cells can be determined as CD19⁺IgM⁻IgA⁻IgD⁻, while IgG-producing B cells can be recognized as CD19⁺IgG⁺.

“Germinal centers” or “germinal centres (GCs)” are sites within lymph nodes and the spleen, wherein mature B cells proliferate, differentiate, and mutate their antibody genes through somatic hypermutation to achieve higher affinity, and switch the class of antibody from IgM to IgG during an immune response. GCs are important in B cell humoral immune response as the center of generation of affinity matured B cells and durable memory B cells. In the GCs, the B cells undergo rapid and mutative cellular division in the dark zone (where they are called centroblasts) and migrate to the light zone (where they are called centrocytes), where they are subject to selection by follicular helper T cells in the presence of follicular dendritic cells. Those selected B cells return to the dark zone to further undergo division and mutation. In the meantime, small amount of memory B cells and plasma cells depart the GCs. In certain embodiments, the in vitro GC like B cells are CD3⁻CD19⁺GL7⁺Fas⁺, which can be identified and sorted by FACS.

The term “T cell” used herein refers to a lymphocyte which is derived from thymus and is mainly involved in cell immunity. Examples of the T cells include a CD4⁺ T cell (T helper cell, T_{H} cell), a CD8⁺ T cell (cytotoxic T cell, CTL), a memory T cell, a regulatory T cell (Treg cell, such as activated Treg and unactivated Treg), an apoptotic T cell, a naïve T cells, or other T cell populations

“T helper cells” are a type of T cells involved in adaptive (that is, tailored to the specific pathogen) immune system via releasing T cell cytokines, thereby suppress or regulate immune responses. T helper cells are involved in B cell antibody class switching, activation and growth of cytotoxic T cells, and maximizing bactericidal activity of phagocytes such as macrophages. Mature T helper cells are CD4 positive and aid the antigen-presenting cells (APCs, such as dendritic cells) to express antigen on MHC class II, via combination of cytokines release and cell to cell interaction (e.g. CD40 (on APC) and CD40L (on T follicular helper cell)). T helper cells can develop into two major subtypes, Th1 and Th2 cells. Th1 helper cells are involved in cellular immune system against intracellular bacteria and protozoa, and are triggered by IL-12 and release IFN-gamma and IL-2. Th1 helper cells help enhance killing efficacy of macrophages, proliferation of CD8⁺ T cells, IgG-production of B cells, and IFN-gamma-secrecting CD4⁺ T cells. Th2 helper cells are involved in humoral immune system against extracellular parasites, and are triggered by IL-4 and IL-2 and release IL-4, IL-5, IL-9, IL-10, IL-13 and IL-25. Th2 helper cells help eosinophils, basophils, mast cells, stimulate B cells to proliferate and to produce antibodies, and IL-4/IL-5-secreting CD4⁺ T cells. T follicular helper cell are found in the periphery within B cell follicles of secondary lymphoid organs such as lymph nodes, spleens and Peyer's patches, and are identified by their constitutive expression of the B cell follicle homing receptor CXCR5. TFH cells trigger the formation and maintenance of germinal centers through the expression of CD40L and the secretion of IL-21 and IL-4 upon cellular interaction and cross-signaling with their cognate follicular (Fo B) B cells.

The term “cytotoxic T cells”, “T-killer cells” or “CTL” used herein is exchangeable and refers to a type of T cells that recognize a specific antigen produced by cancer cells, infected cells by viruses, or cells damaged in other ways. The antigens are brought to the surface of a cell by MHC class I, which is bound by the TCR on cytotoxic T cells in the aid of CD8. Thus, cytotoxic T cells are CD8 positive.

Memory T cells are a subset of T cells that have previously experienced (encountered and responded to) the antigens of cancer cells, bacteria or viruses. The memory T cells can be CD4⁺ and/or CD8⁺ T cells, or memory cytotoxic T cells. Upon re-exposure to an antigen, long-lived memory T cells can mediate a more rapid and more efficient secondary response. This memory function can be provided by CD4⁺ and/or CD8⁺ memory T cells. Long-lived memory T cells are different from effector cells that only have a short life time and usually die after an immune response by activation-inducing cell death (AICD). Between the two cell types, however, there are transitional forms, such as the effector memory cells. Like effector cells, they are able to patrol throughout the body, and exert an effector function upon antigen contact, and they can proliferate and are also more long-lived than effector cells.

“Regulatory T cells” or “Tregs” used herein refers to a subpopulation of T cells that modulate the immune system, maintain tolerance to self-antigens and prevent autoimmune response. Tregs are immnosuppressive and is involved in inhibition of self-reactive immune responses. Tregs are CD4, CLTA4, GITR, neuropilin-1, and CD25 positive. Tregs perform their suppressive function on activated T cells through contact-dependent mechanisms and cytokine production (Fehervari, Z. & Sakaguchi, Curr Opin Immunol 16, 203-8 (2004)). Tregs also modulate immune responses by direct interaction with ligands on dendritic cells (DC), such as CTLA4 interaction with B7 molecules on DC that elicits the induction of indoleamine 2,3-dioxygenase (IDO) (Fallarino, F. et al., Nat Immunol 4, 1206-12 (2003)), and CD40L ligation (Serra, P. et al., Immunity 19, 877-89 (2003)).

“Natural Killer (NK) cells” as used herein refer to lymphocytes which typically have CD16 and/or and/or NCAM and/or CD56 molecules expressed as cell surface markers but which do not express CD3. The NK cells refer to cells present in vivo in a mammal or in vitro in the form of a purified population of cells. NK cells are a type of cytotoxic lymphocyte critical to the innate immune system. The role of NK cells is analogous to that of cytotoxic T cells.

“Dendritic cells (DCs)” are potent antigen-presenting cells (APCs) that process antigen material and present it on the cell surface to the T cells. Upon activation, DCs migrate to the lymph nodes where they interact with T cells and B cells to initiate and shape the adaptive immune response. Human dendritic cells selectively express CD83. DCs have a variety of surface receptors with which they can identify various pathogens. In addition, DCs are able to perceive various endogenous messengers such as cytokines and chemokines, and surface molecules on other cells of the immune system. The DCs process the various incoming signals via intracellular signaling pathways, whereby various differentiation programs are triggered. Dendritic cells are able to initiate primary T cell responses in vitro and in vivo. DCs can be produced ex vivo and loaded with various protein and peptide antigens as well as tumor cell extracts (Nestle, F. et al., Nat. Med., 4:328-332 (1998)). DCs may also be transduced by genetic means to express these tumor antigens as well. DCs have also been fused directly to tumor cells for the purposes of immunization (Kugler, A. et al., Nat. Med., 6:332-336 (2000)).

At least one type of the mononuclear cells, such as B cells, T cells (e.g. T follicular cell), dendritic cells, NK cells, monocytes, can be isolated from the whole blood of a subject, and/or reconstructed from hematopoietic stem cells (HSCs), bone marrow, new born umbilical cord blood (thus called cord blood mononuclear cells (CBMCs)), amniotic fluid, or pluripotent stem cells (hPSCs, comprising both embryonic stem cells (ESCs) and induced pluripotent stem cells (iPSCs)). In certain embodiments, at least one type of the mononuclear cells can be from an adult, adolescent or child.

The hematopoietic stem cells (HSCs) are located in the red bone marrow and generates various type of mature blood cells during the haematopoiesis, including myeloid cells (monocytes, macrophages, neutrophils, basophils, eosinophils, erythrocytes, dendritic cells, and megakaryocytes or platelets) and lymphoid cells (T cells, B cells, and natural killer cells).

“Bone marrow” is the spongy or cancellous, semi-solid tissue in the bone that composed of hematopoietic cells (myeloid and lymphoid lineages), marrow adipose tissue, mesenchymal stem cells (MSCs) and supportive stromal cells. Human bone marrow typically produces around 500 billion blood cells per day that enter into circulation via permeable vasculature sinusoids within the medullary cavity. The lymphoid cells mature in other lymphoid organs, such as thymus.

Umbilical cord blood comprises numerous immunologically immature newborn umbilical cord blood mononuclear cells (UCBMCs) and is also reported a source of hematopoietic stem cells (see Gluckman E et al., Hematopoietic reconstitution in a patient with Fanconi's anemia by means of umbilical-cord blood from an HLA-identical sibling. N Engl J Med. 1989 Oct. 26; 321(17):1174-8.). The mononuclear cells and/or HSCs can be differentiated from human pluripotent stem cells (hPSCs, comprising both human embryonic stem cells (ESCs) and induced pluripotent stem cells (iPSCs)) in vitro, such as primitive hematoendothelial precursors, mature myeloid, erythroid, and lymphoid lineage cells (Melinda K. Hexum et al., In Vivo Evaluation of Putative Hematopoietic Stem Cells Derived from Human Pluripotent Stem Cells, Human Pluripotent Stem Cells, 2011. pp 433-447). Amniotic fluid also contains mononuclear cells and cells with hematopoietic activity (see Ditadi A et al., Human and murine amniotic fluid c-Kit+Lin-cells display hematopoietic activity, Blood. 2009 Apr. 23; 113(17):3953-60).

Activation-induced cytidine deaminase, also known as AICDA and AID, is a 24 kDa enzyme which in humans is encoded by the AICDA gene. AID is a member of the cytidine deaminase family that is involved in somatic hypermutation and class-switch recombination of immunoglobulin genes in B cells and is thought to be the master regulator of secondary antibody diversification. AID generates DNA mutations and turns cytosine to uracil (recognized as thymine during DNA replication), converting C:G to T:A or A:T base pair during germinal center development of B lymphocytes. During somatic hypermutation, the antibody is mutated to generate a library of antibody variants with various affinities.

“Class switch” as used herein, also refers to isotype switching, isotypic commutation or class-switch recombination (CSR). It is a biological mechanism that changes a B cell's production of immunoglobulin (antibodies) from one type to another, such as from the isotype IgM to the isotype IgG and IgE. During this process, the constant-region portion of the antibody heavy chain is changed, but the variable region of the heavy chain stays the same. Since the variable region does not change, class switching does not affect antigen specificity. Instead, the antibody retains affinity for the same antigens, but can interact with different effector molecules (see Honjo et al., Immunity, 1 Jun. 2004, 20(6):659-668). Methods for determination of IgG and IgM and the levels thereof are known in the art, for example, by ELISA using the antibodies specific for the isotypes.

PR domain zinc finger protein 1 is also known as BLIMP-1, which is a transcriptional repressor protein encoded by the PRDM1 gene in humans. BLIMP-1 binds specifically to the PRDI (positive regulatory domain I element) of the beta-interferon (beta-IFN) gene promoter and represses gene expression of beta-IFN. Increased BLIMP-1 protein in B lymphocytes, T lymphocytes, NK cell and other immune cells leads to an immune response through proliferation and differentiation of antibody secreting plasma cells.

The term “hybridoma” used herein refers to a fused hybrid cell in the process of hybridoma technology, which is a method for producing large numbers of monoclonal antibodies. The antibody-producing B cells in response to an immune response are harvested and in turn fused with immortal B cell cancer cells, a myeloma, to produce a hybrid cell line called a hybridoma, which has both the antibody-producing ability of the B-cell and the exaggerated longevity and reproductivity of the myeloma. The hybridomas can be grown in culture, each culture starting with one viable hybridoma cell, producing cultures each of which consists of genetically identical hybridomas which produce one antibody per culture (monoclonal) rather than mixtures of different antibodies (polyclonal). In contrast to polyclonal antibodies, which are mixtures of many different antibody molecules, the monoclonal antibodies produced by each hybridoma line are all chemically identical.

The techniques for selecting “phage display libraries” refers to a method that repertoires of VH and VL genes are separately cloned by polymerase chain reaction (PCR) and recombined randomly in phage libraries, which can then be screened for antigen-binding phage as described in Winter, G. et al., Ann. Rev. Immunol. 12 (1994) 433-455. Phage typically display antibody fragments, either as single-chain Fv (scFv) fragments or as Fab fragments. Libraries from immunized sources (for example the antibody-producing PBMCs made by methods provided herein) provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths, A. D. et al., EMBO J. 12 (1993) 725-734. Finally, naive libraries can also be made synthetically by cloning non-rearranged V-gene segments from stem cells, and using PCR primers containing random sequence to encode the highly variable CDR3 regions and to accomplish rearrangement in vitro, as described by Hoogenboom, H. R. and Winter, G., J. Mol. Biol. 227 (1992) 381-388. Patent publications describing human antibody phage libraries include, for example: U.S. Pat. No. 5,750,373, and US 2005/0079574, US 2005/0119455, US 2005/0266000, US 2007/0117126, US 2007/0160598, US 2007/0237764, US 2007/0292936, and US 2009/0002360. Similar display libraries includes ribosome display, yeast display, bacteria display, baculovirus display, mammal cell display, or mRNA display libraries (see, e.g., U.S. Pat. No. 7,244,592; Chao et al., Nature Protocols. 1:755-768, 2006). These display methods are all conventional techniques in the art, the specific operations thereof can be found in corresponding textbooks or operation manuals, see, e.g. Mondon P et al., Front. Biosci. 13:1117-1129, 2008.

In certain embodiments, a monoclonal antibody is generated via display libraries including the following steps: 1) immunization of single PBMC with an antigen of interest using in vitro immunization as described herein; 2) isolating antigen specific B cell from the above immunized PBMC, and cloning the antibody via PCR and expressing the antibody; 3) obtaining the antibody through various display libraries mentioned above via screening for antibodies with high specificity and affinity.

The term “medium” as used herein refers to a solid, liquid or semi-solid designed to support the growth of microogranisms or cells that supplies the essential nutrients (amino acids, carbohydrates, vitamins, minerals), growth factors, hormones, and gases (CO₂, O₂), and regulates the physio-chemical environment (pH buffer, osmotic pressure, temperature) to the cells. Common medium for culturing mammal cells are known to the art, including but not limited to Dulbecco's Modified Eagel Media (DMEM) (Gibco®), Minimum Essential Medium (MEM), RPMI 1640 Medium (Gibco®), Media 199 (Gibco®), Earles, McCoy's 5A (Hyclone, Thermo Scientific), DMEM/Nutrient Mixture F-12 (DMEM/F12, Gibco®), etc.

In certain embodiments, the stimulants include mitogenic stimulants and antibody producing stimulants. In certain embodiments, the medium further comprises stimulants, including but not limited to CD40- and CD40L-interacting compounds, ICOS- and ICOS-L-interacting compounds, TLR agonists, OX40, OX40L, APRIL (a proliferation-inducing ligand), BAFF, CR2, CXCL9, CXCL12 (SDF-1), CXCL13, CXCL16, Flt-3L, Interleukin-1 (α/β), Interleukin-2, Interleukin-3, Interleukin-4, Interleukin-5, Interleukin-7, Interleukin-10, Interleukin-14, Interleukin-21, SAP (signaling lymphocyte activation molecule [SLAM] associated protein), Staphylococcus A strain Cowan 1 particles (SAC; heat-killed, formalin-fixed), TLR Ligands such as lipopolysaccharide (LPS), different CpG ODNs or Resiquimod (R-848), TSLP, Tumor necrosis factor (TNF) alpha, type I Interferons (e.g. IFN a/(3), type II interferon (e.g. IFNy), lipids, avasimid, EFNB1, EPHB4 (Lu et al., Science, 2017, eaai9264), Plexin B2, semaphoring 4C (Hu et al., Cell Reports, 2017, 19, 995-1007), BLIMP-1, and IRF4. B-cell activation might also be induced via anti-IgG, anti-CD20, and/or anti-CD27 antibodies. In certain embodiments, the stimulants are added to the medium at the beginning of the cultivation, or 1, 2, 3, 4, 5, 6, 7, 8, 9, 10, 11, 12, 13, 14, 15, 16, 17, 18, 19, 20, 21 days later. In certain embodiments, the stimulants are removed from the medium 1, 2, 3, 4, 5, 6, 7, 8, 9, 10, 11, 12, 13, 14, 15, 16, 17, 18, 19, 20, 21 days later. In certain embodiments, two or more of the stimulants exhibit synergistic effects on stimulating in vitro antibody production. For example, in certain embodiments, the two or more of the stimulants comprise ICOS and TLR agonist. In certain embodiments, the two or more of the stimulants comprise CD40L and TLR agonist. In certain embodiments, the two or more of the stimulants comprise ICOS and CD40L. In certain embodiments, the two or more of the stimulants comprises ICOS, CD40L and TLR agonist.

The term “IL2” as used herein refers to interleukin-2, a type of cytokine signaling molecule in the immune system. It is a protein that regulates the activities of white blood cells (leukocytes, often lymphocytes, such as B cells) that are responsible for immunity. IL2 mediates its effects by binding to IL2 receptors, which are expressed by lymphocytes. IL2 is reported to induce proliferation of T cells (Lan, et al., Journal of Autoimmunity, 2008, 31(1):7-12), B cells (Karray, et al., J Exp Med. 1988 Jul. 1; 168(1): 85-94) and dendritic cells. The complete cDNA sequence of IL2 has the GENBANK accession number of AH002842.2 and the amino acid sequence of human IL2 has the GENBANK accession number of AAD48509.1.

The term “IL21” as used herein refers to interleukin-21, which is also a cytokine that costimulates T and natural killer (NK) cell proliferation and function and regulates B cell survival and differentiation and the function of dendritic cells (see Croce et al., J Immunol Res. 2015; 2015: 696578). The complete cDNA sequence of IL21 has the GENBANK accession number of NM_021803.3 and the amino acid sequence of human IL21 has the GENBANK accession number of NP_068575.1.

“Inducible T cell co-stimulator (ICOS)” is also known as “AILIM,” “CD278,” and “MGC39850”. The complete cDNA sequence of ICOS has the GENBANK accession number of NM_012092.3 and the amino acid sequence of human ICOS has GENBANK accession number of NP_036224. ICOS belongs to the CD28 and CTLA-4 cell-surface receptor family and is homologous to CD28 and CTLA-4. It forms homodimers by disulfide linkage and plays an important role in cell-cell signaling, immune responses, and regulation of cell proliferation during the formation of germinal centers, T/B cell collaboration, and immunoglobulin class switching, via the PI3K and AKT pathways. Along with CD28 and CTLA-4, ICOS is expressed on activated CD4 and CD8 T cells and has potential role in regulating the adaptive T cell response, e.g. T cell activation and proliferation. Unlike CD28, which is constitutively expressed on T cells and provides co-stimulatory signals necessary for full activation of resting T cells, ICOS is expressed only after initial T cell activation. ICOS also plays a role in the development and function of other T cell subsets, including Th1, Th2, and Th17. ICOS co-stimulates T cell proliferation and cytokine secretion associated with both Th1 and Th2 cells. ICOS knockout (KO) mice exhibit impaired development of autoimmune phenotypes in a variety of disease models, including diabetes (Th1), airway inflammation (Th2) and EAE neuro-inflammatory models (Th17). In addition to its role in modulating T effector (Teff) cell function, ICOS also modulates T regulatory cells (Tregs). Furthermore, ICOS is expressed at high levels on Tregs, and involves in Treg homeostasis and function (see US patent application US20160304610). The role of ICOS in promoting CD4+ T cell proliferation is implicated to be independent of IL-2 signaling (see Wikenheiser D J and Stumhofer J S, ICOS Co-Stimulation: Friend or Foe? Front Immunol. 2016; 7:304).

Agonist of ICOS (such as ICOSL) binds to the extracellular domain of ICOS, activates the ICOS signaling and thus increases the T cell activation and proliferation.

The term “ICOS ligand (ICOSL)” as used herein is also called “B7H2,” “GL50,” “B7-H2,” “B7RP1,” “CD275,” “ICOSLG,” “LICOS,” “B7RP-1,” “ICOS-L”, and “KIAA0653”, a co-stimulatory molecule of the B7 superfamily, functions as a positive signal in immune response. The complete cDNA sequence of ICOSL has the GENBANK accession number of NM_015259.5 and the amino acid sequence of human ICOSL has the GENBANK accession number of NP 056074.1. ICOSL shares 19-20% sequence identity with CD80/CD86 and is secreted or expressed as a cell surface protein. Human ICOSL has two splice variants (hGL50 and B7-H2/B7RP-1/hLICOS), both of which have identical extracellular domain but differ at the carboxyl-terminal of their cytoplasmic regions. In human, ICOSL is expressed on B cells, dendritic cells, monocytes/macrophages, and T cells. Unlike CD80/CD86, ICOSL does not interact with CD28 or CTLA-4 (CD152) but functions as a non-covalently linked homodimer on the cell surface and binds to ICOS. Human ICOSL is reported to bind to human CD28 and CTLA-4 (see US patent application US20160304610).

ICOS/ICOS-L's interaction is involved in T cell-mediated immune responses in vivo. Furthermore, in vivo deficiency in ICOS causes impaired germinal center (GC) formation (reduction in the numbers and size of the GCs), defect in isotype class switching in T cell-dependent B cell responses and defects in L-4 and IL-13 production (see Khayyamian et al., ICOS-ligand, expressed on human endothelial cells, costimulates Th1 and Th2 cytokine secretion by memory CD4 T cells, PNAS, Vol. 9, No. 9, 2002, 6198-6203). In the GC, long-lived plasma cells (LLPCs) and memory B cells (MBCs) undergo class-switching and somatic hypermutation to increase antibody affinity.

In certain embodiments, cultivating PBMC in the presence of ICOS can enhance the total amount of antibody or antigen-binding fragment thereof produced by the PBMCs.

Agonist of ICOS can be screened by determination of their affinity and specificity of binding. The method for determining the affinity and specificity of binding, such as competitive and non-competitive binding assay are known in the art, including ELISA, RIA, flow cytometry, etc. The effects of ICOS agonists can be determined by a functional assay detecting the T cell activation by ICOS. The T cell activation can be measured via detection of CD4+ T cell proliferation, cell cycle progression, release of cytokines, such as IL-2, upregulation of CD25 and CD69, etc.

The ICOS agonists include compounds or proteins, such as an agonist antibody JTX-2011 (Jounce Therapeutics Inc) and GSK3359609 (GSK), and the antibodies described in US patent application US20160304610, US 20170174767, as well as WO 2012/131004.

CD40L, as used herein, is also called CD40 ligand or CD154, a protein that is primarily expressed on activated T cells (its expression has since been found on a wide variety of cells, including platelets, mast cells, macrophages, basophils, NK cells, B lymphocytes, as well as non-hematopoietic cells) and is a member of the TNF superfamily of molecules. It binds to CD40 on antigen-presenting cells (APC) and acts as a costimulatory molecule that is particularly important on a subset of T cells called T follicular helper cells (TFH cells). On TFH cells, CD40L promotes B cell maturation and function by engaging CD40 on the B cell surface and therefore facilitating cell-cell communication. The complete cDNA sequence of CD40L has the GENBANK accession number of NM_000074.2 and the amino acid sequence of human CD40L has the GENBANK accession number of NP 000065.1.

The phrase “B-cell activating factor” or “BAFF” or “Baff” as used herein refers to a tumor necrosis family ligand, e.g., a TNF family ligand. BAFF is expressed on the surface of a cell and serves as a regulatory protein involved in interactions between membrane surface proteins on immune cells, e.g., B cells. Secreting BAFF is efficient B cell growth factor, and help B cell to proliferate and function as a co-stimulator. It is reported that BAFF is critical to the survival of antibody-secreting cell from memory cells (Avery D V et al., J Clin Invest, 2003, 112:286-97).

“OX40L” is the ligand for OX40 (CD134) and is expressed on cells such as DC2s (a subtype of dendritic cells) enabling amplification of Th2 cell differentiation. OX40L has also been designated CD252 (cluster of differentiation 252). It has been reported that OX40 co-express with ICOS in T follicular helper cells (Tfh) and affect interaction between Tfh cells-B cells in germinal center (GC), thereby affecting the B cell development and differentiation and maturation of plasma cells in the GC.

The term “Toll-like receptor (TLR)” is a family of proteins that play a key role in the innate immune system (non-specific immunity). They are single, membrane-spanning, non-catalytic receptors usually expressed on sentinel cells such as macrophages and dendritic cells, that recognize structurally conserved molecules derived from microbes. Beside the extracellular and transmembrane domain, a TLR comprises a cytoplasmic Toll-interleukinl receptor-resistance (TIR) domain. Once these microbes have breached physical barriers such as the skin or intestinal tract mucosa, they are recognized by TLRs, which activate immune cell responses. The TLRs recognize highly conserved structural motifs, i.e. pathogen-associated molecular patterns (PAMPs) that are exclusively expressed by microbial pathogens, such as lipopolysaccharide (LPS) from gram-negative bacteria and lipoteichoic acid (LTA) from gram-positive bacteria and flagellin, etc, or danger-associated molecular patterns (DAMPs) that are endogenous molecules released from necrotic or dying cells. Many tumor cells undergo necrosis mediated by the immune system and may lead to further activation of an inflammation response via TLRs. The human TLR family includes TLR1, TLR2, TLR3, TLR4, TLR5, TLR6, TLR7, TLR8, TLR9, and TLR10, which are expressed on a variety of immune cell types. Mouse TLR family includes TLR1-9 and TLR 11-13.

Among human antigen presenting cells (APCs), TLR7, 9, and 10 are expressed on plasmacytoid dendritic cells (pDCs), whereas all TLRs except TLR9 are expressed on myeloid derived DCs (mDCs). In human adaptive immune system, TLR1, 2, 3, 4, 5, 7, and 9 are expressed on T cells, and TLR5 and 8 are expressed on regulatory T cells (Treg), a cell type critical to the maintenance of immune homeostasis. Finally, activated and memory B cells express significant levels of TLR1, 6, 7, 9, and 10 but low levels of TLR2 (see Deng Sl et al., Recent advances in the role of toll-like receptors and TLR agonists in immunotherapy for human glioma, Protein Cell 2014, 5(12):899-911). TLRs 1, 2, and 4-6 are expressed on the cell surface and sense bacterial, fungal, and protozoal products, whereas TLRs 3 and 7-9 are expressed in endosomes and sense viral nucleic acids (see Maisonneuve C et al., Unleashing the potential of NOD- and Toll-like agonists as vaccine adjuvants. Proc Natl Acad Sci USA. 2014 Aug. 26; 111(34):12294-9).

“Toll-like receptor ligand” as used herein refers to agonists or antagonists of Toll-like receptor. In certain embodiments, the TLR ligand is an agonist, such as pathogen-associated molecular patterns (PAMPs). Examples of the TLR agonist that activates TLR includes, but not limited to imiquimod, GS-9620 (Gilead, see Roethle et al, 2013), compound 32 (GSK2245035, GSK, see Biggadike et al, 2016), and resiquimod (R848), imidazoquinolines, nucleic acids comprising an unmethylated CpG dinucleotide (e.g. ODN2216) and poly I:C, monophosphoryl lipid A (MPLA) or other lipopolysaccharide derivatives, single-stranded or double-stranded RNA, flagellin, muramyl dipeptide, TSLP, Tumor necrosis factor (TNF) alpha, type I Interferons (e.g. IFN α/β), type II interferon (e.g. IFNy), lipids, avasimid, EFNB1, EPHB4, Plexin B2, semaphoring 4C, BLIMP-1, and IRF4. (see J. Med. Chem. Roethle et al, 2013. Identification and Optimization of Pteridinone Toll-like Receptor 7 (TLR7) Agonists for the Oral Treatment of Viral Hepatitis. J. Med. Chem. Biggadike et al, 2016. 59, 1711-1726. Discovery of 6-Amino-2-{[(1S)□1-methylbutyl]oxy}-9-[5-(1-piperidinyl)pentyl]-7,9-dihydro□8H□purin-8-one (GSK2245035), a Highly Potent and Selective Intranasal Toll-Like Receptor 7 Agonist for the Treatment of Asthma.)

TLR agonists specific to the TLR types are reported, for example, BCG (TLR1, 2, 4, and 6), lipopeptides (TLR1, 2, and 6), monophosphoryl lipid A (MPL), LPS, RC529, AS01, AS02, AS04 and glucopyranosyl lipid adjuvant (GLA-SE) (TLR4), poly(I:C) (TLR3), flagellin (TLR5), single stranded and R484/resiquimod (TLR7 and TLR8) or double stranded (ds) RNA (TLR3), imiquimod and Type 1 interferon (TLR7) and DNA containing the CpG motif, AS 15, and IC31 (TLR9). Endogenous molecules released from stressed or dead cells such as heat shock proteins (HSP; TLR2 and TLR4) and high mobility group box 1 (HMGB 1; TLR2 and TLR4) are also reported important TLR agonists (see Deng S1 et al., Recent advances in the role of toll-like receptors and TLR agonists in immunotherapy for human glioma, (see Protein Cell 2014, 5(12):899-911; Zhang W W and Matlashewski G, Immunization with a Toll-Like Receptor 7 and/or 8 Agonist Vaccine Adjuvant Increases Protective Immunity against Leishmania major in BALB/c Mice, INFECTION AND IMMUNITY, August 2008, p. 3777-3783; Gauwelaert N D et al., The TLR4 Agonist Vaccine Adjuvant, GLA-SE, Requires Canonical and Atypical Mechanisms of Action for TH1 Induction, PLoS One. 2016 Jan. 5; 11(1):e0146372; Maisonneuve C et al., Unleashing the potential of NOD- and Toll-like agonists as vaccine adjuvants. Proc Natl Acad Sci USA. 2014 Aug. 26; 111(34):12294-9).

Activation of TLRs occurs mainly through homodimerization of the TLR upon ligand binding, while TLR2 forms heterodimers with both TLR1 and TLR6. The TLR agonists can activate both the innate and adaptive immune systems. The activated TLRs signal through one of two different pathways, myeloid differentiation factor 88 (MyD88)-dependent (all TLRs except TLR3) and MyD88-independent (TLR3). The former involves MyD88 and TIRAP, and leads to early activation of NF-κB, MAPK, and transcription of pro-inflammatory cytokines, chemokines, and cytosolic enzymes, while the latter involves adaptors TRIF and TRAM, and results in the activation of late phase NF-κB and the interferon (IFN) regulatory factors responsible for type I IFN expression (see Deng S1 et al., Recent advances in the role of toll-like receptors and TLR agonists in immunotherapy for human glioma, Protein Cell 2014, 5(12):899-911; MacLeod H and Wetzler L M. T cell activation by TLRs: a role for TLRs in the adaptive immune response. Sci STKE. 2007 Sep. 4; 2007(402):pe48.).

The present disclosure discovers that in vitro activation of ICOSL and CD40L increases in vitro production of antibody (e.g. IgM and IgG) up to 1.2, 1.5, 2, 2.5, 3, 4, 5, 6, 7, 8, 9, 10, 15, 20, 25-fold or more, as compared with that of CD40L alone.

The present disclosure discovers that in vitro activation of the toll-like receptor 7 (by adding TLR agonist, e.g. imiquimod) increases in vitro production of antibody (e.g. IgM and IgG) up to 1.2, 1.5, 2, 2.5, 3, 4, 5, 6, 7, 8, 9, 10, 15, 20, 25-fold or more, as compared with that of CD40L alone.

The present disclosure discovers that in vitro activation of ICOS increases in vitro production of antibody (e.g. IgM and IgG) up to 1.2, 1.5, 2, 2.5, 3, 4, 5, 6, 7, 8, 9, 10, 15, 20, 25-fold or more, as compared with that of CD40L alone.

The term “in vitro immunization” refers to the induction of a humoral response in vitro, i.e. the in vitro production of antigen-specific human antibodies which results from the recognition of said antigen by the immunoglobulins expressed at the surface of naive human B lymphocytes cultured, in vitro, with the antigen. An in vitro immunization (IVI) method in the art for producing a monoclonal antibody or antigen-binding fragment thereof is described by Tomimatsu et al. (Tomimatsu et al. Methods Mol Biol. 2014; 1060:297-307), which generally includes: isolating human PBMC from healthy donor by centrifugation, collecting lymphocytes; treat the isolated PBMCs (i.e. lymphocytes) with LLME to remove immunosuppressive cells and enable the in vitro sensitization with antigen; incubating the LLME treated cells with antigen of interest in the presence with cytokines (IL-2, IL-4), D-type and K-type CpG-ODN and cultured for a week; the antibody produced by the cells are detected and quantified with immunoassay, such as ELISA; isolating the RNA of the antibody produced by lymphocytes and constructing the antibody encoding sequences into phage display library.

The term “antigen-presenting cell” or APC, is intended to mean a cell expressing one or more molecules of the class I and class II major histocompatibility complex (MHC) (class I and class II HLA molecules in humans) and capable of presenting antigens to CD4⁺ T and CD8⁺ T lymphocytes specific for this antigen. As antigen-presenting cells, mention may in particular be made of dendritic cells (DCs), peripheral blood mononuclear cells (PBMCs), monocytes, macrophages, B lymphocytes, lymphoblastoid lines, and genetically modified human or animal cell lines expressing class I and class II MHC molecules, in particular HLA I and HLA II molecules.

The term “antigen cell surface molecule” is intended to mean a molecule expressed at the surface of antigen-presenting cells. The term “surface molecule specific for antigen-presenting cells” is intended to mean a surface molecule expressed only on APCs or a molecule expressed essentially on antigen-presenting cells, i.e. a surface molecule expressed on APCs and also on a very limited number of cells other than APCs, and as a result having a high specificity of expression for APCs, i.e. a molecule virtually specific for APCs.

The term “chimeric antigen receptor” or “CAR” as used herein refers to an artificially constructed hybrid protein or polypeptide containing an antigen binding domain of an antibody (e.g., a single chain variable fragment (scFv)) linked to T-cell signaling or T-cell activation domains (see, e.g., Kershaw et al., supra, Eshhar et al., Proc. Natl. Acad. Sci. USA, 90(2): 720-724 (1993), and Sadelain et al., Curr. Opin. Immunol. 21(2): 215-223 (2009)). CARs are capable of redirecting T-cell specificity and reactivity toward a selected target in a non-MHC-restricted manner, taking advantage of the antigen-binding properties of monoclonal antibodies. The non-MHC-restricted antigen recognition confers T-cells expressing CARs on the ability to recognize an antigen independent of antigen processing, thus bypassing a major mechanism of tumor escape. In addition, when expressed in T-cells, CARs advantageously do not dimerize with endogenous T-cell receptor (TCR) alpha and beta chains.

In certain embodiments, the CAR sequence comprises an antigen binding domain, such as VH and VL gene segments of the antibody prepared according to the methods provided herein, and a T-cell signaling domain, which comprises, e.g. a hinge-CH2-CH3, a transmembrane domain and one or more cytoplasmic signaling domains. In certain embodiments, a transmembrane domain includes, but not limited to, transmembrane domains from CD8 alpha, CD4, CD45, PD1, and CD152. In certain embodiments, the cytoplasmic signaling domains includes but not limited to intracellular co-stimulatory signaling domains from CD28, CD54 (ICAM), CD134 (OX40), CD137 (41BB), CD152 (CTLA4), CD273 (PD-L2), CD274 (PD-L1), and CD278 (ICOS) and a primary signaling domain from CD3 zeta or FcR gamma.

The present disclosure further provides uses of the CAR so produced in immunotherapy, such as in chimeric antigen receptor T-cell therapy (CAR-T).

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Materials and Methods

Materials:

### LSM Lymphocyte Separation Medium (MP, cat.V0111A)

LLME: L-leucyl-L-leucine methyl ester (BacheM, cat.G-2550.0001)

### Ham's F-12 Nutrient Mixture (Gibco, cat. 11765047)

Heparin anticoagulation tube (BD, cat.367878)
Disposable blood collecting needle (BD, cat.367237)
IL2, Interleukin-2, lymphokine, TCGF (sinobiological, cat. 11848-HNAY1-50)
BCGF-1, BCGF1, BSF-1, BSF1, IL-4, Interleukin-4 (sinobiological, cat.GMP-11846-HNAE-100)
CD154, CD40 Ligand (sinobiological, cat. 10239-HO1H-50)
OX40L (sinobiological, cat. 13127-H04H-100)
Human ICOS Ligand/B7-H2/ICOSLG (Histag) (sinobiological, cat.11559-HO8H-100)
Human ICOS/AILIM/CD278 Protein (His & Fc Tag) (sinobiological, cat. 10344-H03H-100)
Human Interleukin-21/IL21 (sinobiological, cat.GMP-10584-HNAE-20)
Human BLyS/TNFSF 13B/BAFF (sinobiological, cat.10056-HNCH-5)
Ephrin-B 1 (sinobiological, cat. 10894-H08H)
Goat anti-Human IgG-Fc (HRP) (sinobiological, cat.SSA001-1)
Goat anti-Human IgM mu chain (HRP) (Abcam, cat.ab97205)

### GlutaMAX™ Supplement (Gibco, cat.35050-061)

### MEM NEAA (Gibco, cat.11140-050)

Sodium pyruvate (Gibco, cat. 11360-070)

### DMEM (no Glutamine, no Sodium Pyruvate, no HEPES) (Gibco, cat.11960-051)

### Penicillin-Streptomycin, Liquid (Gibco, cat. 15140122)

### RPMI 1640 Medium (Gibco, cat.21875091)

DAPI (4′,6-diamidino-2-phenylindole; stock: 5 mg/ml in dH2O; Thermo Fisher, cat. no. D1306)
TMB substrate (TIANGEN, cat.PA107-01)

### FBS (GIBCO, cat. 10099141)

### PBS (8117158)

E6446 dihydrochloride (MCE,cat. HY-12756A)
Anti-Human CD3 PE-Cy7 (eBioscience,cat. BG-05121-77-100)
Anti-Human CD21 PE (eBioscience,cat. 85-12-0219-42)
Mouse Anti-Human CD35-FITC (eBioscience,cat. 05-9600-02)
Anti-Human CD19 PerCP-Cy5.5 (eBioscience,cat. BG-11211-70-100)
Imaging reader (Biotek, cat.Cytation 5)
96 well Elisa plate (Corning, cat.9018)

Methods:

Preparation of Human Peripheral Blood Mononuclear Cells (PBMC)

Culture medium was prepared for PBMCs: (RPMI1640: DMEM: Ham's F12=1:1:2) (eRDF) supplemented with 10% FBS. Fresh PBMC was harvested from several healthy donors (about 40 ml/time/person). PBMC was separated by density-gradient centrifugation as previously described in human monoclonal antibody book. Cell number was counted with a hemocytometer.

In Vitro Immunization (IVI)

Diluted the washed PBMC with 10% FBS+eRDF, adjust cell density to 1*10\hat{ }7 cells/ml. Treated cell with 0.25 mM LLME for about 20 min. Discarded supernatant, and re-suspended cell with 10% FBS+eRDF. Adjusted cell density to 9*10\hat{ }5 cells/ml. Transferred cell suspension into 96 well plate, and added 2 μg/ml antigen, 10 ng/ml IL2, 2 μg/ml CD40L, 2 μg/ml ICOS, TLR7 agonist (commercial imiquimod or synthesized), respectively. Cultured the tissue for 7 days at 37° C., 5% CO2. Changed half of the medium and added a cytokine/activator cocktail containing 10 ng/ml IL2 and 50 ng/ml IL21 on day 7. Cultured the cells for 7-21 days at 37° C., 5% CO₂. Collected the supernatant on day 7, day 14, or day 21 for analysis of antibody production by ELISA, whereas the pellets are used for testing gene expression by PCR or RT-PCR. The collected cells were also tested for FACS analysis. In all the experiments, the antigen OVA or TrkA was added at 2 μg/ml, and IL2 at 10 ng/ml and IL21 at 50 ng/ml, where IL2, IL21 and the antigen alone or in combination were added.

Exemplary IVI steps with addition of ICOSL:

1. Prepared PBMCs and treat them with LLME.
2. Cultured and amplified the treated PBMCs with IL2 (10 ng/ml) for 2-3 weeks.
3. Collected the cells and plate them into 96-well plate at a density of 6*10⁴/well.
4. Treated the cells with the mixture including ICOSL (50 ng/ml), CD40L (50 ng/ml), IL21 (50 ng/ml) and antigen for 14 days, and changed half of the medium in day 7.
5. After removing the mixed factors, the single factor IL2 plus antigen were added into well to stimulate IgG production for 7-14 days.
6. The IgG and IgM level was examined with ELISA assay.

For human hybridoma, the method further comprises the below steps:

7. Collected the treated PBMCs and fused them with human myeloma cell line. Screened the cell line with hypoxanthine-aminopterin-thymidine (HAT) medium.
8. Cultured the hybridoma for 14 days and changed half of the medium in day 7.
9. Harvested the supernatant medium and examine the antibody titer with ELISA assay.

Measurement of the Antibody Level after Incubation with Stimulants

After day7 or day14 with addition of cytokines or stimulant factors and antigens, supernatants were harvested and added to antigen (OVA or TrkA, respectively)-coated plates. After 2 hr incubation, a HRP-conjugated anti-human IgG or anti-human IgM was added, the amount of antigen-specific antibody was measured using TMB as substrate. The data represents the mean of 2 replicates; error bars represent SD. One representative data of 3 separate experiments is shown.

Flow Cytometry

We analyzed stained cells on an airell (BD) and processed flow cytometry data with FlowJo software (Tree Star). PBMCs were collected into Snap-lock microtubes. For analysis of T cells or B cells, tubes were kept at 4° C. unless mentioned otherwise. After centrifugation, cells were washed and resuspended in PBS. For analysis of T follicular helper cells, PBMCs were stained with antibodies of CD3-FITC (BD), CD4-PerCP-Cy™5.5 (BD), CXCR5-PE/Cy7 (Biolegend), and CD45RA-PE(eBioscience), respectively. T follicular helper cells were identified with CD3⁺CD4⁺CXCR5⁺CD45RA⁻. For analysis of GC like B-cells, PBMC was stained with antibodies of CD19-PE (eBioscience), GL7-Alexa Fluor 488 (eBioscience), Fas-APC(eBioscience). GC like B-cells were defined as CD19⁺GL7⁺Fas⁺.

Reverse Transcription PCR

Quantitative RT-PCR was carried out with a BioRad iCycler and the 2-(ΔΔCT) method was used to calculate relative mRNA expression levels normalized to GAPDH.

Enzyme-Linked Immunosorbent Assay

Plates were coated with antigen at 5 μg/ml overnight at 4° C. and washed them in PBST (containing 0.5% Tween-20). The plates were blocked with 5% BSA before addition of cell culture supernatants and horseradish peroxidase (HRP)-conjugated detection antibodies (dilutions: 1 in 2,500 for HRP-conjugated IgG-specific antibody (Jackson) and HRP-conjugated IgM-specific antibody. TMB substrates solution was used for measurement.

Enzyme Linked Immunospot Assay (ELISpot)

Nitrocellulose-backed 96-well MAHAS4510 plates (Millipore) were coated overnight at 4° C. with (5 μg/mL) in 50 mM sodium bicarbonate buffer (pH 9.6). Plates were washed and blocked for 2 h at 37° C. with 10% fetal calf serum in RPMI1640. PBMCs were seeded at 3*10\hat{ }5 cells/well and incubated for 24 h at 37° C. Spot-forming cells (SFCs) were then detected using 2,000-fold diluted goat anti-human IgG antibody conjugated with horse radish peroxidase and incubate for 2 hr at 37° C. Ab binding was evaluated by the addition of TrueBlue substrate solution substrate (KPL, Gaithersburg, Md.).

Statistical tests with appropriate underlying assumptions on data distribution and variance characteristics were used. Except when noted other-wise, Two-way ANOVA were used to compare endpoint means of different groups. Regression and graphing were performed with Prism6 (GraphPad).

### Example 2: IL2 Stimulate the Proliferation of the PBMCs

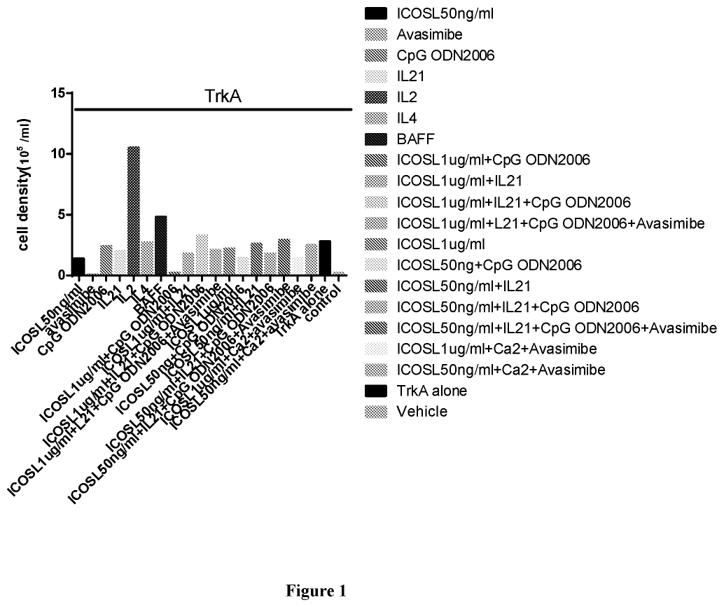

PBMC includes antibody-producing B cell, T cell and dendritic cell populations. The expansion of these cells can form the germinal-center like structure in vitro. Results are shown in FIG. 1. In the Figure, “Control” represents cells without antigen or any stimulants. All other columns represent cells treated with the antigen TrkA together with various factors. Note that IL2 is the most potent stimulant that promotes cell proliferation.

### Example 3: ICOSL is a Key Stimulant that Induces the Antibody Production

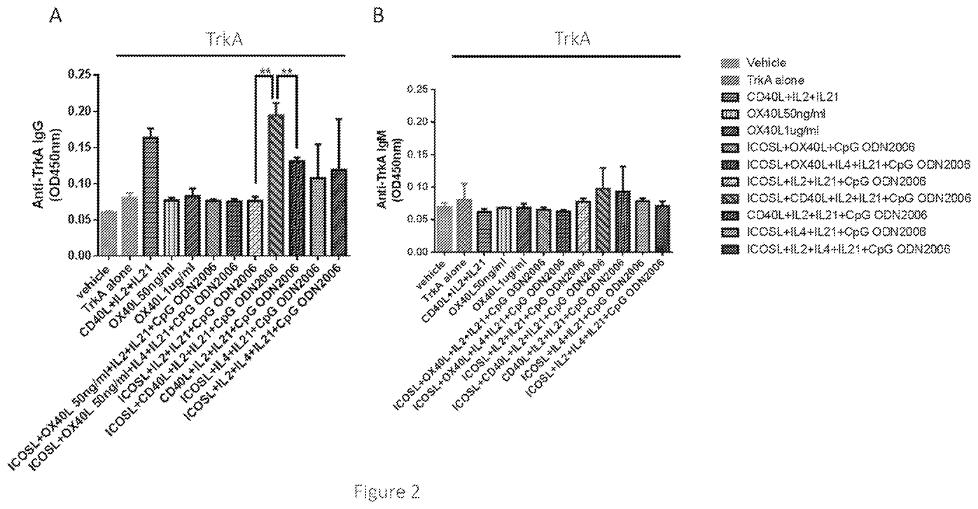

In the amplified PBMCs, ICOSL were added together with the antigen TrkA and other stimulants to the medium. We found human antibody (IgM & IgG) synthesis/production is enhanced within the B cells by the stimulant mixture including ICOSL, together with other critical ingredients CD40L, IL2, IL21 and CpG ODN after culture of 10-14 days. ICOSL is also a key stimulant that induce the highest antibody level among all the stimulants. Results are shown in FIG. 2A-2B, which indicated that ICOSL and CD40L synergistically enhance the IgG production, rather than ICOSL or CD40L alone.

### Example 4: IL21 Promotes the Class Switch from IgM to IgG

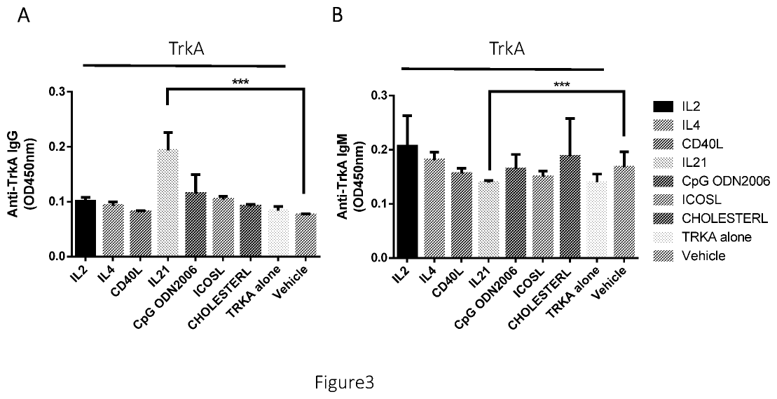

After 14-day culture with the mixture of ICOSL, CD40L, IL21 and CpG ODN, the IgG production was increased but IgM production was slightly decreased in the culture with the stimulant IL21 only. Results are shown in FIG. 3A-3B.

### Example 5: Effects of ICOS

1. ICOS is Also a Key Stimulant that Increase the Antibody Production

To further test effects of other cytokine or stimulants on in vitro antibody production, ICOS (55 nM) was added to the IVI system in the presence of the antigen OVA (2 μg/ml) or TrkA (2 μg/ml). PBMCs (1.5*10\hat{ }5 cell/well, 96 well plate) were incubated with or without OVA/IL2/Il21 (IL2+IL21=basic), OVA/IL2, OVA/IL21, OVA alone, OVA/IL2/IL21/ICOS, OVA/IL2/IL21/CD40L, TrkA/IL2/IL21/ICOS, TrkA/IL2/IL21/CD40L, and vehicle (PBS), respectively. *, p<0.05 for cells stimulated with CD40L vs. cells stimulated with ICOS, antigen is OVA. ****, p<0.0001 for cells stimulated with CD40L vs. cells stimulated with ICOS. The antigen was TrkA.

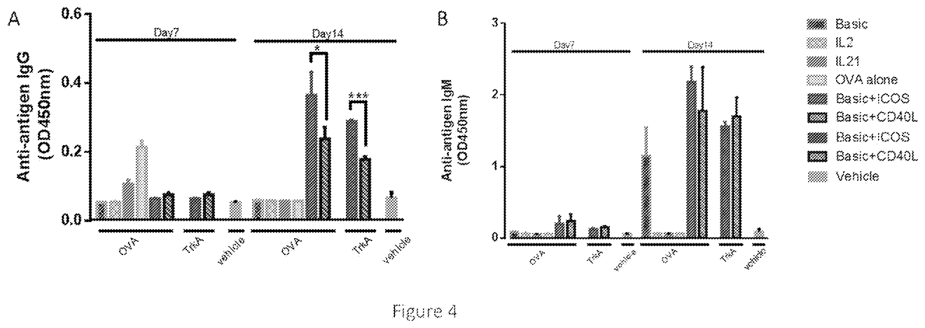

The results in FIGS. 4A and 4B showed that in vitro stimulation with ICOS enhances the production of antibody (both IgM and IgG) against OVA or TrkA. Note that for either OVA or TrkA as an antigen, ICOS is more effective than CD40L in stimulating the production of the antibody IgG (about 1.5 fold higher).

2. ICOS Promotes Accelerated Generation of the GC-Like Phenotype (CD19⁺GL7⁺) B Cells

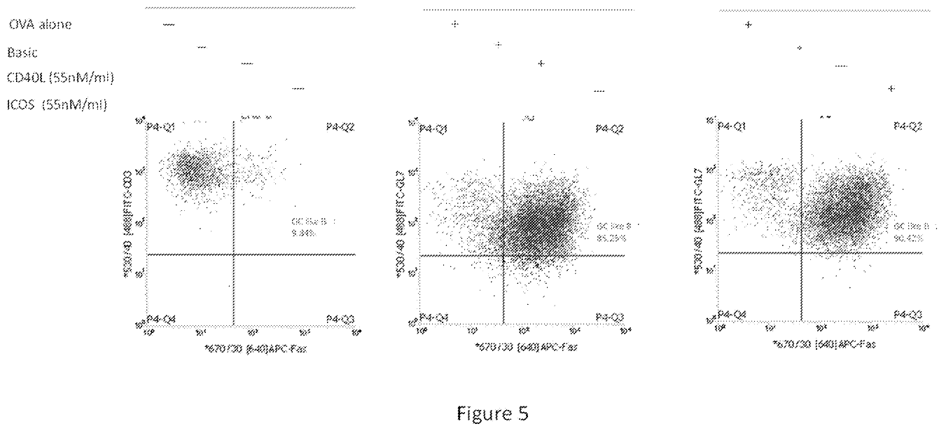

After FACS sorting, GC like B cells are gated as CD3⁻, CD19⁺, GL7⁺, Fas⁺ cells. Numbers within the gates represent the GC like B cells of different groups. As shown in FIG. 5A to 5C, the PBMCs incubated without any stimulant was sorted on day 0, the ratio of GC like B cells is about 9.84% (FIG. 5A). The PBMCs immunized with the antigen OVA (2 μg/ml), treated with CD40L in a cocktail of IL2 (10 ng/ml) and IL21 (50 ng/ml) (basic), and sorted on day14. The ratio of GC like B cells is about 85.25% (FIG. 5B). Similarly, the PBMCs treated with ICOS. The ratio of GC like B cells is about 90.42% (FIG. 5C).

### Example 6: Effects of Toll-Like Receptor Agonist

1. TLR Agonist is Another Key Stimulant that Induces the Antibody Production.

To further test effects of other cytokine or molecules on antibody production, a synthesized TLR7/8 agonist (50 nM and 500 nM) was added to the IVI system in the presence of antigen OVA (2 μg/ml). PBMCs (1.5*10\hat{ }5 cell/well, 96 well plate) were incubated without or with OVA/IL2/Il21, OVA/IL2, OVA/IL21, OVA alone, OVA/IL2/IL21/CD40L, OVA/IL2/IL21/the synthesized TLR7/8 agonist (50 nM), OVA/IL2/IL21/the synthesized TLR7/8 agonist (500 nM), and vehicle, respectively. *, p<0.05 for cells stimulated with the synthesized TLR7/8 agonist (50 nM) vs. cells stimulated with CD40L cells. ****, p<0.0001 for cells stimulated with the synthesized TLR7/8 agonist (500 nM) vs. stimulation with CD40L cells.

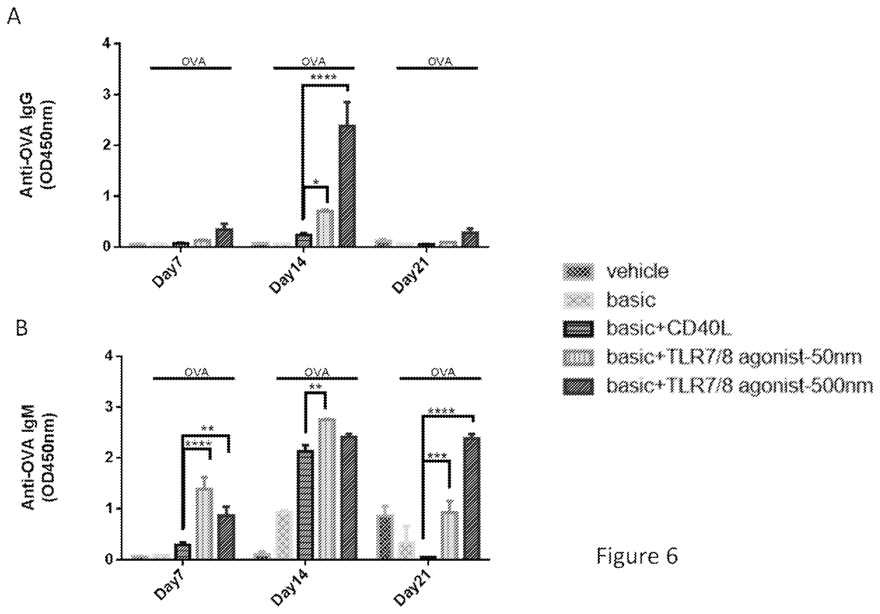

The results in FIGS. 6A and 6B show that TLR7/8 agonist was much more potent in stimulating the production of anti-OVA antibody. Note that for IgG antibodies, the TLR7/8 agonist was more effective at 14 days in vitro (about 3.5 and 10.0 fold higher than CD40L with 50 nM and 500 nM of the TLR agonist, respectively) than at 7 days and 21 days in vitro (FIG. 6A). For IgM antibodies, TLR7 agonist was effective at 7, 14, 21 days in vitro. At 7 and 21 days, TLR7/8 agonist was far more effective than CD40L. Thus, to selectively stimulate IgG, the optimal time for TLR7/8 treatment is 7 days.

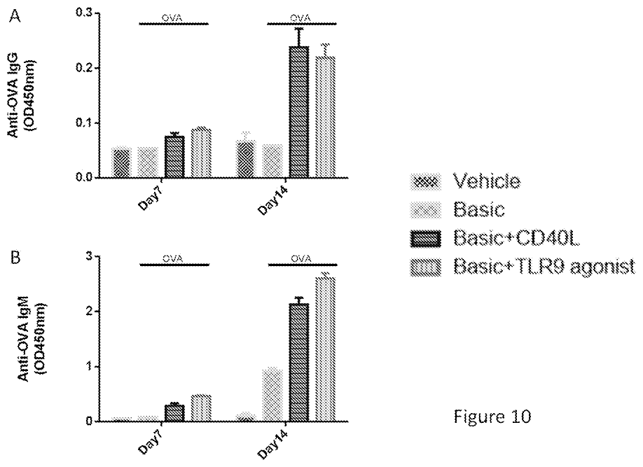

Similar tests were also performed using the TLR9 agonist CpG ODN (2 g/ml). FIG. 10 shows that at day 14, CpG ODN elicited similar effects as CD40L in stimulating the production of anti-OVA antibodies (for both IgG and IgM).

PBMCs (4*10\hat{ }5 cell/well, 48 well plate) derived from different donors (donor 1 and donor 2) were incubated with or without OVA/IL2/IL21/CD40L, OVA/IL2/IL21/synthesized TLR agonist (50 nM), OVA/IL2/IL21/synthesized TLR7/8 agonist (500 nM), and vehicle (PBS), respectively.

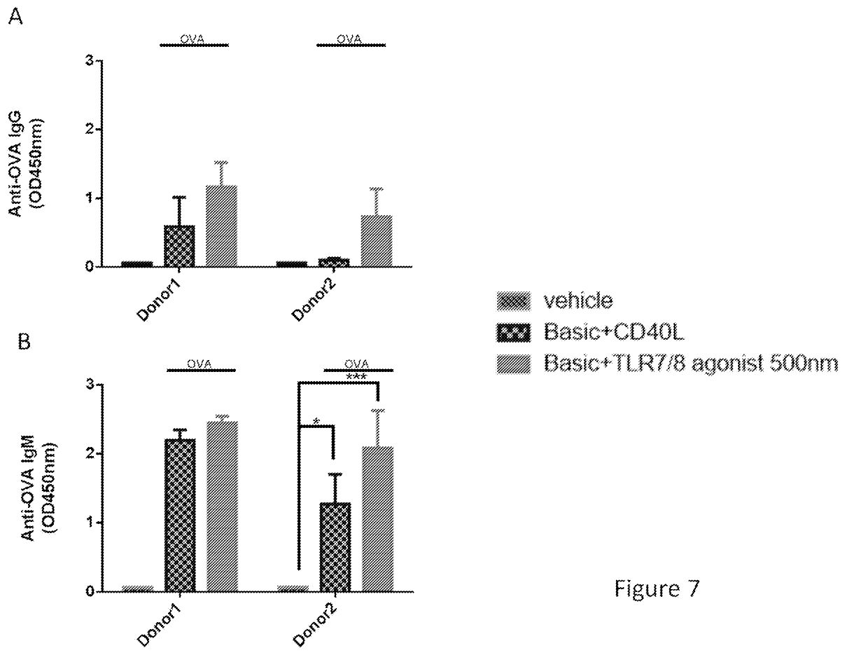

FIGS. 7A and 7B represent data from 2 different PBMC donors showing that the synthesized TLR7/8 agonist was either similar to (donor 1) or more effective than (donor 2) CD40L in stimulating antibody production. Error bars represent SD. *, p<0.05 for cells stimulated with CD40L (2 μg/ml). ****, p<0.0001 for cells stimulated with the synthesized TLR7/8 agonist (500 nM).

2. Expression of AID and BLIMP-1 were Increased by TLR7 or TLR7/8 Agonists

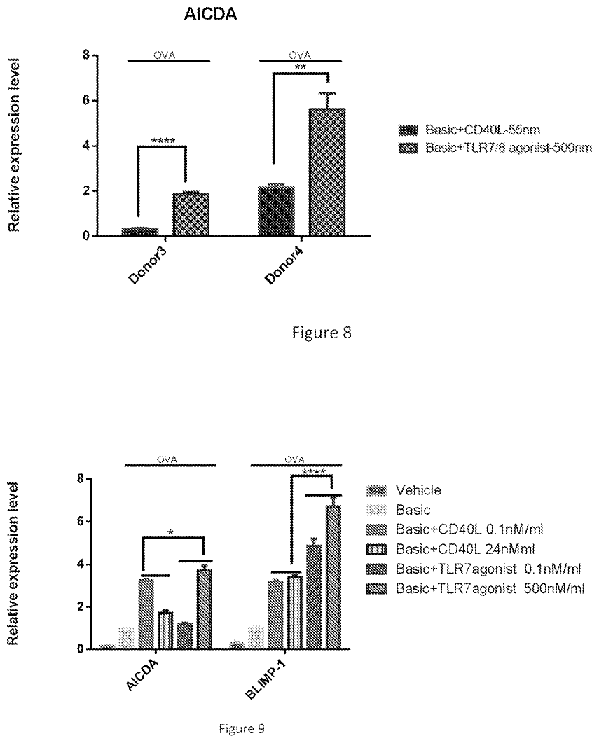

AID is known to be involved in B cell affinity maturation by inducing hyper-mutation in antibody genes. Expression of BLIMP-1 represents the proliferation and differentiation of active B cell. To test the effect of TLR agonists on the expression of AID, mRNA levels of AICDA (that encoding AID) after treatment with the synthesized TLR7/8 agonist (500 nM), in comparison with that of CD40L (55 nM), was determined by quantitative RT-PCR. PBMCs were collected from two different donors (see FIG. 8, donor 3 and donor 4). PBMCs (4*10\hat{ }5 cell/well, 48 well plate) were incubated with or without OVA/IL2/IL21/CD40L, OVA/IL2/IL21/synthesized TLR7/8 agonist (500 nM), OVA/IL2/IL21, and vehicle (PBS), respectively. Glyceraldehyde 3-phosphate dehydrogenase expression in PBMCs after incubation with OVA/IL2/IL21 was used for normalization. AID and BLIMP-1 expression in PBMCs after incubating with OVA/IL2/IL21 was used as control. The error bars represent SD. **, p<0.005 for cells stimulated with the synthesized TLR7/8 agonist vs. cells stimulated with CD40L, for PBMCs from donor 4. **** p<0.0001, cells stimulated with the synthesized TLR7/8 agonist vs. cells stimulated with CD40L, for PBMC from donor 3.

To test the effect of TLR7 agonist imiquimod on the expression of AICDA and BLIMP-1, mRNA levels of AICDA and BLIMP-1 after IVI in the presence of the TLR7 agonist imiquimod (500 nM), was determined by quantitative RT-PCR, in comparison with that of CD40L (FIG. 9). PBMCs were collected from a healthy donor. PBMCs (1.5*10\hat{ }5 cell/well, 96 well plate) were incubated with or without OVA/IL2/IL21, OVA/IL2/IL21/CD40L (0.1 nM), OVA/IL2/IL21/CD40L (24 nM), OVA/IL2/IL21/imiquimod (0.1 nM), OVA/IL2/IL21/imiquimod (500 nM), and vehicle (PBS), respectively. *, p<0.05 for cells stimulated with TLR7 agonist (500 nM) vs. cells stimulated with CD40L (0.1 nM), for AICDA mRNA. ****, p<0.0001 for cells stimulated with TLR7 agonist (500 nM) vs. cells stimulated with CD40L (24 nM), for AICDA mRNA.

Both FIG. 8 and FIG. 9 show that TLR7 agonist and synthesized TLR7/8 agonist are far superior to CD40L in stimulating the expression of AICDA and BLIMP-1. Also indicated in FIG. 8 and FIG. 9 is the superior ability of TLR7 in inducing enriched antibody variants via hypermutation, and higher affinity of the antibody, as compared with CD40L. Hence, TLR7 is implicated as a potential stimulant to promote generation of antibodies against an antigen with lower immunogenicity.

3. Synergistic Effects Between the Synthesized TLR7/8 Agonist and TLR9 Antagonist E6446.

PBMCs (3×10⁵ cells/well, 48 well plate) from 2 healthy human were pre-incubated with 0.02 uM E6446, 0.2 uM E6446, 10 uM E6446, OVA+IL2+IL21 and medium for 1 h. Then wells containing E6446 (E6446 0.02 uM, E6446 0.2 uM and E6446 10 uM) and OVA+IL2+IL21 were stimulated with 500 nM synthesized TLR7/8 agonist, while OVA+IL2+IL21 and medium were added to the low-stimulated control (basic) and unstimulated control (vehicle), respectively. After 7 days or 14 days, supernatants were removed for test.

E6446 dihydrochloride (E6446-HCL), a synthetic nucleic acid-sensing TLRs antagonist. It's IC50 for CpG2006 (TLR9 agonist) was in the range of 0.01-0.03 uM (data not shown). But it required 2-8 uM (a 100-fold higher concentration) to inhibit the effect of R848 (TLR7/8 agonist) (data not shown).

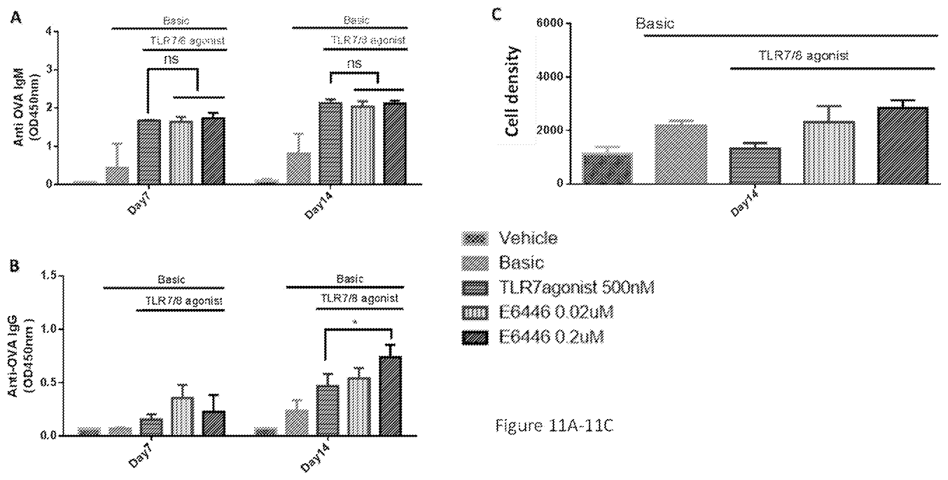

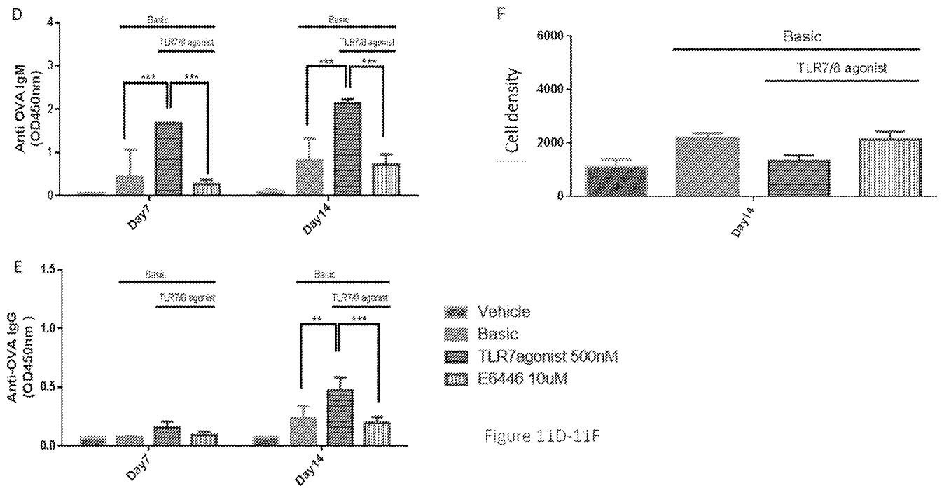

The antibody responses of peripheral blood mononuclear cells (PBMCs) was triggered by the synergistic interaction of stimulants and a cocktail of stimulants containing 10 ng/ml IL2 and 50 ng/ml IL21 (see FIGS. 11A-11G).

FIGS. 11A-11C show that the synergy between TLR7/8 agonist and low concentration of TLR9 antagonist E6446 (e.g. 0.02 uM and 0.2 uM) was only observed for IgG but not IgM production in 14-day but not in 7-day old cultures, indicating that low concentration of E6446 promotes the effects of TLR7/8 agonist on antibody IgG production.

FIGS. 11D-11F show that antigen-specific IgG and IgM responses were remarkably boosted by the synthesized TLR7/8 agonist, however, the response was blocked by addition of TLR7/8 antagonist (10 uM E6446 dihydrochloride). High concentration of TLR9 antagonist E6446 (10 uM) reverses the effects of TLR7/8 agonist in both 7-day and 14-day cultures, for both IgG and IgM.

FIG. 11G shows that PBMCs treated with the synthesized TLR7/8 antagonist exhibited a dramatic CD21 reduction in vitro, revealing that the CD21-sub-type is inhibited by high concentration of E6446. In contrast, stimulation of PBMCs with TLR7/8 agonist after blocking TLR9 (0.02 uM and 0.2 uM E6446 dihydrochloride, respectively) in vitro resulted in a significant increase in IgG responses and cell activity, indicating that TLR7/8 agonist facilitates the generation of dendritic cells, which is partially reversed by high concentration of TLR9 antagonist E6446.

4. Stimulation of IL2, IL21 or Combination on Further Enhancement by Stimulants on IgG Production

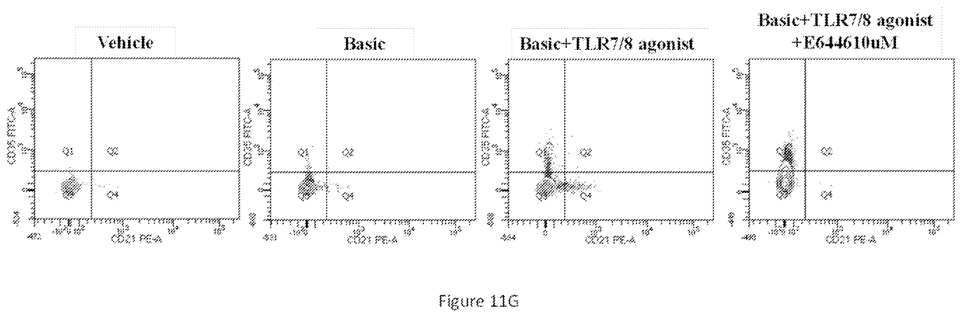

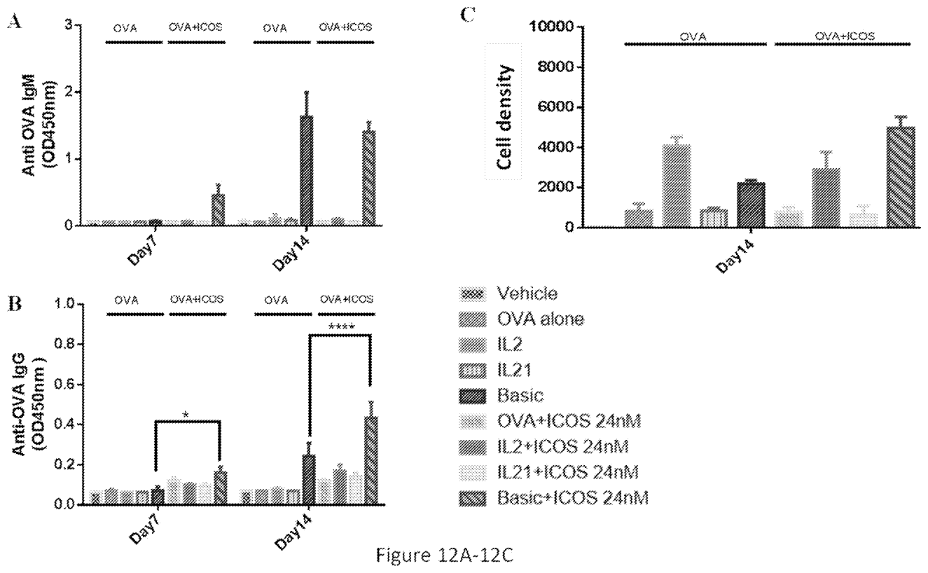

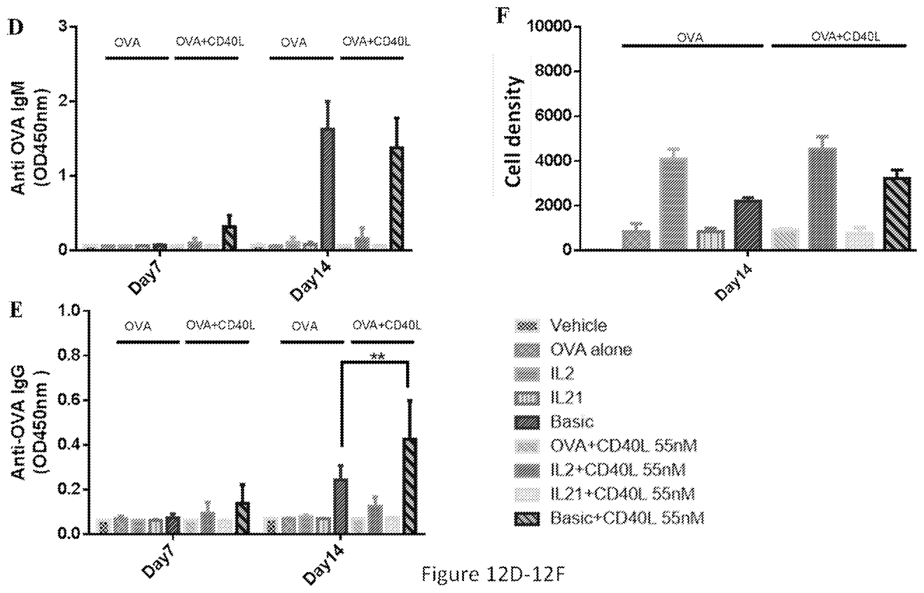

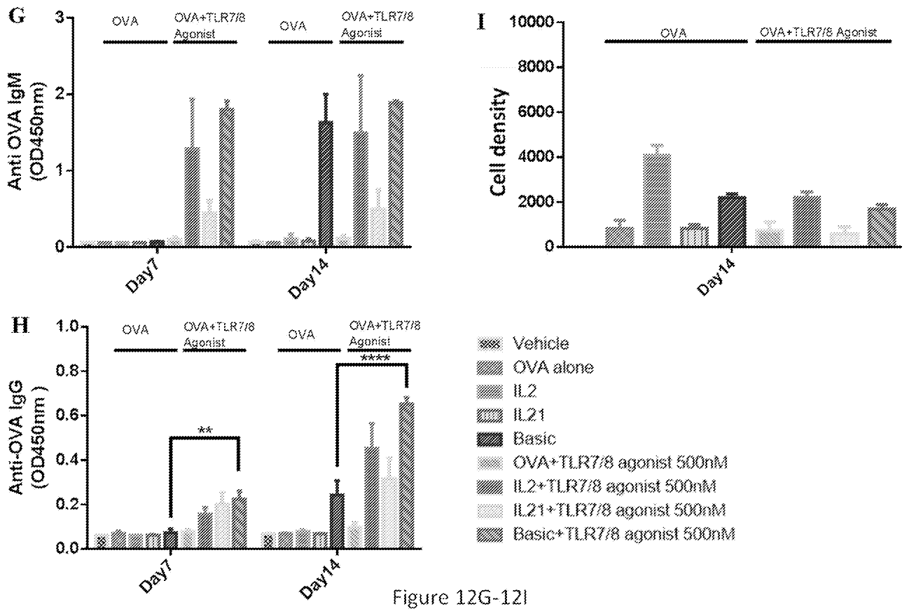

PBMCs (3×10⁵ cells/well, 48 well plate) from 2 healthy human were incubated with or without 4 ug/ml OVA, 10 ng/ml IL2, 50 ng/ml IL21, 24 nM ICOS, 55 nM CD40L, 500 nM synthesized TLR7/8 agonist and their combinations. After 7 days or 14 days, supernatants were removed for test. IL2 and IL21 co-stimulated with ICOS, CD40L, or synthesized TLR7/8 agonist respectively resulted in synergistic, complimentary effects on enhanced IgG production (see FIGS. 12A-12I).

5. Dose-Dependent Effects of Stimulants on IgG and IgM Production

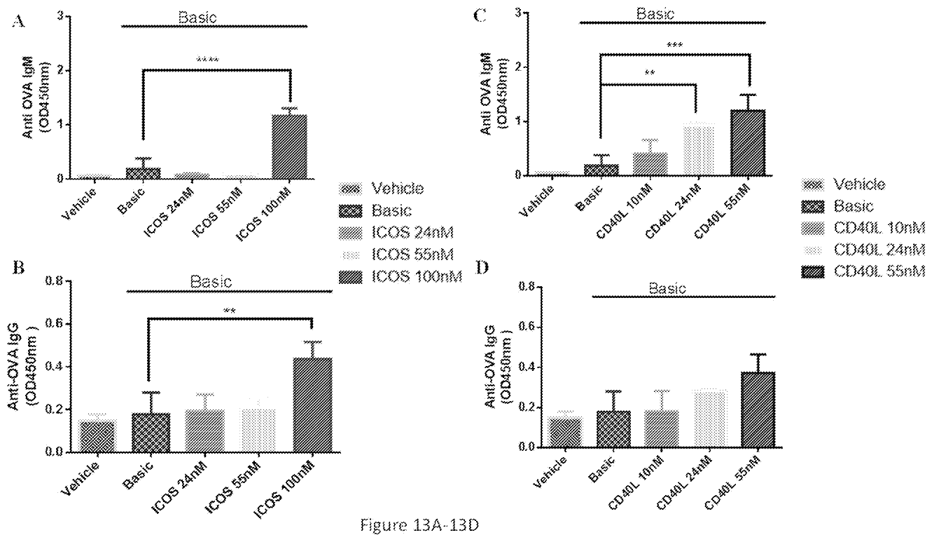

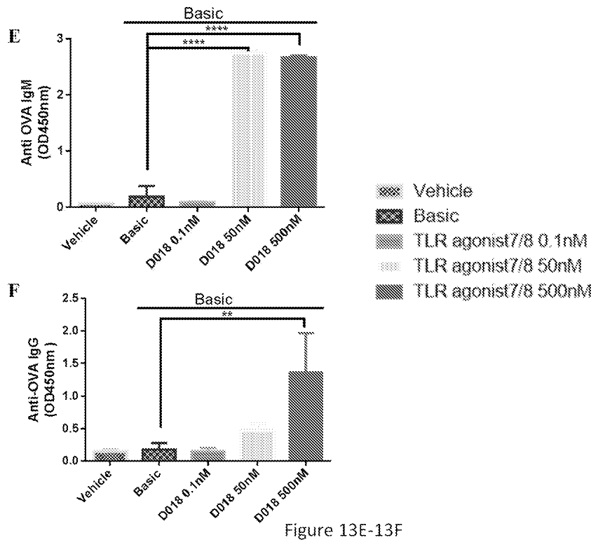

PBMCs (3×10⁵ cells/well, 48 well plate) from 2 healthy subjects were incubated with or without 4 ug/ml OVA, 10 ng/ml IL2, 50 ng/ml IL21, ICOS (24 nM, 55 nM, 100 nM), CD40L (10 nM, 24 nM, 55 nM), synthesized TLR7/8 agonist (0.1 nM, 50 nM, 500 nM) and their combinations. After 7 days or 14 days, supernatants were removed for test. FIGS. 13A-13F show that ICOS, CD40L, TLR7/8 agonist regulated IgG and IgM responses in a dose-dependent manner.

Co-stimulation of PBMCs in vitro with 10 ng/ml IL2 and 50 ng/ml IL21 enhanced both antigen-specific IgG and IgM responses, specifically after exposure to stimulants such as ICOS, CD40L and synthesized TLR7/8 agonist, respectively. Furthermore, the data also indicated that ICOS, CD40L and TLR7/8 agonist act as dose-dependent regulators for antibody production.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for producing an antibody or antigen-binding fragment thereof comprising a step of cultivating PBMCs in a medium comprising at least one of CD40L, ICOSL, ICOS and TLR agonist.

2. (canceled)

3. (canceled)

4. (canceled)

5. (canceled)

6. The method of claim 1, wherein the medium further comprises IL2 and/or IL21.

7. The method of claim 1, wherein the TLR agonist is a TLR7 agonist, a TLR8 agonist or a TLR9 agonist.

8. The method of claim 1, wherein the TLR agonist is a TLR7 and TLR8 (TLR7/TLR8) agonist.

9. (canceled)

10. (canceled)

11. (canceled)

12. (canceled)

13. (canceled)

14. The method of claim 1, wherein the PBMCs comprises B cells, T follicular cells and dendritic cells.

15. The method of claim 1, wherein the medium further comprises an antigen.

16. (canceled)

17. (canceled)

18. (canceled)

19. (canceled)

20. (canceled)

21. The method of claim 1, wherein the at least one of CD40L, ICOSL, ICOS and TLR agonist induces enhancement of antibody production by the PBMCs, B cell activation and differentiation, and/or B cell maturation in the PBMCs.

22. The method of claim 1, further comprising a step of isolating an antibody secreted from the cultivated PBMCs.

23. The method of claim 22, further comprising obtaining a nucleic acid sequence encoding a variable region of the antibody.

24. The method of claim 23, further comprising introducing the nucleic acid sequence into a host cell under a condition suitable for expressing the antibody or antigen-binding fragment thereof.

25. The method of claim 1, wherein the at least one of CD40L, ICOSL, ICOS and TLR agonist is present at a concentration of at least 0.5 ng/ml, and/or at least 0.1 nM.

26. The method of claim 6, wherein IL2 is present at a concentration of at least 0.5 ng/ml, and/or IL21 is present at a concentration of at least 0.5 ng/ml.

27. (canceled)

28. The method of claim 25, wherein the at least one of CD40L, ICOSL, ICOS and TLR agonist is present for at least 1 day.

29. The method of claim 26, wherein the IL2 is present for at least 1 day, and/or the IL21 is present for at least 1 day.

30. (canceled)

31. The method of claim 1, wherein the antibody is monoclonal antibody, polyclonal antibody, or full human antibody.

32. A method for inducing proliferation of PBMCs, B cell activation and differentiation, and/or B cell maturation, comprising a step of cultivating PBMCs in a medium comprising IL2.

33. (canceled)

34. A method for promoting class switch in an antibody-producing PBMC to produce IgG, comprising a step of cultivating the antibody-producing PBMC in a medium comprising IL21.

35. (canceled)

36. (canceled)

37. (canceled)

38. The method of claim 1, further comprising
obtaining a nucleic acid sequence encoding a variable region of the antibody; and optionally
introducing the nucleic acid sequence into a host cell under a condition suitable for expressing the antibody or antigen-binding fragment thereof.

39. The method of claim 38, further comprising
isolating the antibody secreted by the host cell.

40. (canceled)

41. A method for producing a chimeric antigen receptor (CAR), comprising a step of expressing a first nucleic acid operably linked to a second nucleic acid, wherein the first nucleic acid encodes an antigen binding domain derived from the antibody or antigen-binding fragment thereof produced according to the method of claim 1, and wherein the second nucleic acid encodes a T-cell signaling domain.

42. (canceled)

43. (canceled)
